(19) United States
(12) Patent Application Publication
(10) Pub. No.: US 2025/0108081 A1
(43) Pub. Date: Apr. 3, 2025

(54) Improved Probiotic Potency of the Yeast Saccharomyces Boulardii

(71) Applicants: VIB VZW, Gent (BE); Katholieke Universiteit Leuven, Leuven (BE)

(72) Inventors: Johan Thevelein, Blanden (BE); Bruna Trindade De Carvalho, Leuven (BE); Maria Foulquié Moreno, Brussel (BE); Paul Vandecruys, Heusden-Zolder (BE); Jeroen Raes, Berchem (BE); Geert Huys, Aarsele (BE); Severine Vermeire, Leuven (BE); Sara Deleu, Leuven (BE)

(21) Appl. No.: 18/833,223

(22) PCT Filed: Jan. 26, 2023
(86) PCT No.: PCT/EP2023/051941

§ 371 Date: Jul. 25, 2024

(30) Foreign Application Priority Data
Jan. 27, 2022 (EP) 22153700.4
Apr. 28, 2022 (EP) 22170421.6

(51) Int. Cl. A61K 36/064; A61P 1/00; C12N 1/18; C12N 9/02; C12N 9/10; C12N 9/22; C12N 15/11; C12N 15/90

(52) U.S. Cl. CPC A61K 36/064; A61P 1/00; C12N 1/18; C12N 9/0008; C12N 9/1025; C12N 9/22; C12N 15/11; C12N 15/905; C12Y 102/01004; C12Y 203/03001; C12N 2310/20

(57) ABSTRACT

The present invention relates to the field of probiotics, more particularly to the probiotic yeast Saccharomyces boulardii. The present invention provides genetic engineering approaches such as the use of gene deletions and the use of chimeric gene constructs to develop yeast strains with enhanced production of acetic acid. In addition, the invention also relates to the use of such yeast strains for the production of dietary supplements or pharmaceutical compositions to improve gastrointestinal comfort.

### CROSS-REFERENCE TO RELATED APPLICATIONS

This application is a national phase entry under 35 U.S.C. § 371 of International Patent Application PCT/EP2023/051941, filed Jan. 26, 2023, designating the United States of America and published in English as International Patent Publication WO 2023/1442740n Aug. 3, 2023, which claims the benefit under Article 8 of the Patent Cooperation Treaty to European Patent Application Serial No. 22153700.4, filed Jan. 27, 2022, and to European Patent Application Serial No. 22170421.6, filed Apr. 28, 2022, the entireties of which are hereby incorporated by reference.

### INCORPORATION BY REFERENCE

The ST.26 XML Sequence listing named “10577 US—Sequence Listing”, created on Jan. 1, 2026, and having a size of 32,768 bytes, is hereby incorporated herein by this reference in its entirety.

### FIELD OF THE INVENTION

The present invention relates to the field of probiotics, more particularly to the probiotic yeast Saccharomyces boulardii. Even more particularly the present invention relates to enhanced probiotic potency of S. boulardii. The present invention provides genetic engineering approaches such as the use of gene deletions and chimeric gene constructs to develop yeast strains with enhanced production of acetic acid. In addition, the invention also relates to the use of such yeast strains for the production of dietary supplements or pharmaceutical compositions to improve gastrointestinal comfort.

### BACKGROUND

The human gut microbiome comprises thousands of species with high interindividual variability (Tap et al 2009 Environ Microbiol 11; Pasolli et al 2019 Cell 176). A healthy microbiome is key to maintaining and restoring good health and its disruption through antibiotic treatment is associated with elevated risk of diabetes (Kilkkinen et al 2006 Diabetologia 494), Crohn's disease (Hviid et al 2010 Gut Pathog 60), overweight (Azad et al 2014 Intern J Obesity 38) and even asthma and allergies (Metsälä et al 2014 Clinical & Experimental Allergy 457; Ni et al 2019 BMC Pediatrics 19; Metsälä et al 2013 Epidemiology 20). Probiotics—defined as live microorganisms that confer beneficial effects on their hosts when administered in drug-like quantities—have been used for a long time to modulate the microbiota and assist in recovery from certain diseases. The most frequently used probiotics are lactic acid bacteria, such as Lactobacillus spp. and Bifidobacterium spp., ingested with fermented foods (Plaza-Diaz et al 2019 Adv Nutri 10). Among eukaryotic microorganisms, Saccharomyces cerevisiae var. boulardii (S. boulardii) is probably the best known, studied and commercialized probiotic organism. The use of yeast as probiotic has a clear advantage over existing bacterial probiotics because of its natural resistance to bacterial antibiotics. Probiotic yeasts are therefore advised for patients suffering from antibiotic induced diarrhea (More and Swidsinski 2015 Clin Exp Gastroenterol 14; Czerucka et al 2007 Aliment Pharmacol Ther 26). S. boulardii is also commonly used in the treatment of pathogen-induced diarrhea and other gastrointestinal (GI) disorders, with positive results in both Crohn's disease and ulcerative colitis (Czerucka et al 2007 Aliment Pharmacol Ther 26; McFarland 2010 World J Gastroenterol 16; Guslandi et al 2003 Eur J Gastroenterol Hepatol 15; Girardin and Seidman 2011 Dig Dis 29).

Despite the molecular evidence of a close relationship with S. cerevisiae, S. boulardii is considered as a different species and exhibits some unique metabolic and physiological attributes. S. boulardii shows much better tolerance to acidic conditions akin to that of the gastric milieu when compared to S. cerevisiae (Cascio et al 2013 BMC Microbiol 13; Edwards-Ingram et al 2007 Appl Environ Microbiol 73; Fietto et al 2004 Can J Microbiol 73). It possesses an enhanced ability for pseudohyphal switching (Edwards-Ingram et al 2007 Appl Environ Microbiol 73) and thrives better at 37° C. (Fietto et al 2004 Can J Microbiol 73) which is an important requirement for probiotic microorganisms. It produces elevated levels of metabolites such as myo-inositol, 2-ethoxyindole and 4-hydrophenylethanol when compared to S. cerevisiae (MacKenzie et al 2008 Yeast 25). Furthermore, S. boulardii lacks the ability to sporulate (van der Aa Kuhle & Jespersen 2003 Syst Appl Microbiol 26; Edwards-Ingram et al 2007 Appl Environ Microbiol 73), a trait commonly present in most S. cerevisiae strains (Tomar et al 2013 PLoS One 8). A recent breakthrough was the discovery of high acetic acid production by S. boulardii, a unique phenotypic property that is absent in S. cerevisiae (WO2019/053218A1; Offei et al 2019 Genome Res 29). Acetic acid or acetate is a crucial substrate in the gut for the bacterial butyrate production, a SCFA known to limit intestinal inflammation by promoting regulatory T cells (Cushing et al 2015 Clin Transl Gastroenterol 6). Furthermore, high acetic acid production contributes to the reduction of gastrointestinal lumen pH, a well-known mechanism of action against Salmonella, Vibrio cholerae and Blastocystis (Kaźmierczak-Siedlecka et al 2020 Curr Microbiol). The acetic acid production ability of S. boulardii could be attributed to a non-functional SDH1 gene (Offei et al 2019). Interestingly, all S. boulardii strains comprise a unique point mutation in SDH1 while SDH1 loss-of-function mutations could not be found in any of the sequenced S. cerevisiae strains. Moreover, it was demonstrated that acetic acid production could be further increased by deleting the Whi2 function in S. boulardii. The S. boulardii isolates Sb.P and Sb.A, which are homozygous for the recessive mutation whi2^{s270*}, are able to accumulate unusually high amounts of acetic acid and could strongly inhibit bacterial growth (WO2019/053218A1). However, the homozygous whi2^{s270*} mutation also leads to acetic acid sensitivity and acid sensitivity in general. This is a major disadvantage since probiotics have to survive the acid environment of the stomach before colonizing or passing through the gut. It furthermore implicates that acetic acid production in the Sb.P and Sb.A strains can hardly be increased because the viability of the strains is inversely correlated with acetic acid production. It would thus be advantageous to engineer high acetic acid producing S. boulardii strains that are not sensitive to low pH.

### SUMMARY

We have now engineered new S. boulardii strains producing even higher levels of acetic acid than the Sb.P strain while keeping the same tolerance towards low pH as the non-engineered parent strains. One of these newly engineered strains, named ENT3, is also able to accumulate much higher acetic acid concentrations when growing on low glucose levels, in contrast to the ENT wild type parent and Sb.P strains. It is also demonstrated that the medium supernatant of ENT3 is more potent than the one of Sb.P against all tested bacterial strains.

Hence, it is an object of the application to provide a Saccharomyces boulardii yeast having a compromised or partially abolished Cit1 or Ach1 function. In one embodiment, said compromised or partially abolished Cit1 function is due to the presence of a homozygous loss-of-function CIT1 mutant allele. In another embodiment, said compromised or partially abolished Ach1 function is due to the presence of a homozygous loss-of-function ACH1 mutant allele.

It is another object of the application to provide a S. boulardii yeast comprising a homozygous loss-of-function CIT1 and/or ACH1 mutant allele. In one embodiment, said loss-of-function allele is a CIT1 and/or ACH1 deletion.

In another object of the application, any of the S. boulardii yeasts described above is provided wherein ALD4 expression is statistically significantly enhanced. Said increase in expression is compared to an isogenic control S. boulardii yeast not engineered for increased ALD4 expression. In one embodiment, said increased ALD4 expression is due to the presence in the S. boulardii yeast of at least one, more particularly at least 2, even more particularly at least 4 chimeric gene constructs comprising at least one ALD4 allele for ALD4 overexpression. In a particular embodiment, said chimeric gene construct comprises the TEF1 promoter driving the expression of the ALD4 allele.

It is yet another object of the application to provide a Saccharomyces boulardii yeast producing acetic acid at 37° C. in low glucose medium of about 20 mM glucose. In a particular embodiment, said S. boulardii yeast produces at least 0.25 g/l, 0.5 g/l, 1 g/l or 1.25 g/l acetic acid. In one embodiment, said S. boulardii yeast permanently produces acetic acid at 37° C. in low glucose medium of about 50 mM glucose. In a particular embodiment, said S. boulardii yeast produces at least 0.5 g/l, 1 g/l, 1.5 g/l, 2 g/l, 2.5 g/l, 3 g/l or at least 3.5 g/l acetic acid. In a particular embodiment, said S. boulardii yeast produces at least 0.5 g/l, 1 g/l, 1.5 g/l, 2 g/l, 2.5 g/l, 3 g/l or at least 3.5 g/l acetic acid. In another embodiment, said S. boulardii yeast has an ALD4 expression level in the exponential growth phase that is at least 2-fold, 2.5-fold or at least 3-fold higher compared to a control S. boulardii yeast. In another embodiment, said S. boulardii yeast has an ALD4 expression level in the stationary or early stationary growth phase that is at least 1.25-fold, 1.5-fold or at least 1.8-fold higher compared to a control S. boulardii yeast. In another embodiment, said S. boulardii yeast comprises at least two chimeric gene constructs comprising at least one ALD4 allele. In a more particular embodiment, said S. boulardii strain comprises at least four chimeric gene constructs comprising at least one ALD4 allele. In an even more particular embodiment, said chimeric gene construct comprises the TEF1 promoter driving the ALD4 expression.

In a specific embodiment of the application, any S. boulardii yeast herein described is not the Sb.P or Sb.A strain. In another specific embodiment, said S. boulardii yeast comprises at least one WHI2 wild-type allele. In yet another specific embodiment, said S. boulardii yeast comprises a homozygous or hemizygous SDH1 mutant allele, more particularly an SDH1 loss-of-function allele, even more particularly the SDH1 mutant allele is as depicted in SEQ ID No. 11. In yet another specific embodiment, said S. boulardii yeast is the CNCM 1-475 or the ENT strain.

It is also an object of the application to provide a dietary supplement or pharmaceutical composition comprising any of the S. boulardii yeast herein disclosed. The S. boulardii yeasts herein disclosed are furthermore provided for use as a medicament, more particularly for use in the treatment or prevention of gastrointestinal disorders, even more particularly for use in the treatment or prevention of gastrointestinal disorders caused by Clostridia, Citrobacter, Escherichia, Salmonella, Shigella, Klebsiella, Vibrio, Blastocystis, Enterobacter or combinations thereof. This is similar as saying that a method is provided for maintaining or improving the health of the gastrointestinal tract in a human or animal, said method comprising administering to said human or animal, a dietary supplement or pharmaceutical composition comprising any of the S. boulardii yeasts disclosed herein. More particularly, said maintaining or improving the health of the gastrointestinal tract comprises reducing the number of pathogenic bacteria found in the gut and/or the faeces of said human or animal. Even more particularly, said pathogenic bacteria are selected from the group consisting of Clostridia, Citrobacter, Escherichia, Salmonella, Shigella, Klebsiella, Vibrio, Blastocystis, Enterobacter and mixtures thereof.

In a final aspect, the use of any of the S. boulardii yeast described herein as a live probiotic additive to foodstuff and/or feedstuff is provided, as well as the use of said S. boulardii yeasts for the production of acetic acid.

### BRIEF DESCRIPTION OF THE FIGURES

FIGS. 1A and 1B. Evaluation of acetic acid accumulation in the Sb.P background by modification of selected targets (FIG. 1A) overexpression and (FIG. 1B) deletion. Cells were propagated in YPD 2% at 37° C., 200 rpm for 72 h. Results are the mean of three biological replicates.

FIGS. 2A and 2B. Evaluation of acetic acid accumulation in the ENT background caused by modification of selected targets (FIG. 2A) overexpression and (FIG. 2B) deletion. Cells were propagated in YPD2% at 37° C., 200 rpm for 72 h. Results are the mean of three biological replicates. (The ENT ach1ΔΔ strain used in Panel B is colony 2; see below)

FIGS. 3A and 3B. Effect of ALD4 overexpression in the ENT1 strain on acetate accumulation. (FIG. 3A) acetate accumulation as a function of time. (□) ENT wild type, (∘) Sb.P, (●) ENT1, (▪) ENT2, (▴) ENT3 (FIG. 3B) acetate accumulation at 72 h. Cells were propagated in YPD2% at 37° C., 200 rpm for 72 h. Results are the mean of three biological replicates. Different letters indicate a significant difference between groups (p-value<0.001 for all groups compared to each other using one way ANOVA).

FIG. 4. Effect of ALD4 overexpression on acetate accumulation at low glucose concentration. Cells were propagated in YPD 0.9% (50 mM) at 37° C., 200 rpm for 72 h. Results are the mean of three biological replicates.

FIGS. 5A-5E. Comparison of Sb.P (●) and ENT3 (602) performance for acetate accumulation at different glucose levels. (FIG. 5A) 5 mM, (FIG. 5B) 20 mM, (FIG. 5C) 50 mM, (FIG. 5D) 75 mM and (FIG. 5E) 110 mM. Cells were propagated in at 37° C., 200 rpm for 72 h. Results are the mean of two biological replicates.

FIG. 6. Effect of ADH1 deletion on acetate, ethanol and glycerol accumulation. ENT3 is represented in black and ENT3 adh1ΔΔ in grey. Cells were propagated in YPD2% at 37° C., 200 rpm for 48 h.

FIG. 7. Agar well diffusion assay. Nutrient Schaedler agar was inoculated with each one of the tested pathogenic bacteria strains. Wells were punched into agar and filled with: 100 μg/mL ampicillin (control), pure acetic acid (3, 6, 9, 12 g/L, in YP pH 4.2) or cell-free supernatant from Sb.P and ENT3 cultures. Two sets of plates were prepared, one of which was incubated inside an anaerobic jar. Plates were incubated at 37° C. for 24-48 h.

FIG. 8. Viability after exposure to simulated gastric acid. Tolerance to the low pH of simulated gastric acid was determined by comparing CFU of cells exposed to saline (NaCl 0.5%) at pH 6.8 or pH 1.7. Cells were incubated for 3 h at 37° C. Different letters indicate significant differences between groups (P<0.05 for all groups compared to each other using one way ANOVA).

FIG. 9 shows the disease activity determined by the area under the curve for mice treated with the negative controls PBS or Baker's yeast, with the SDH1 strain, ENT strain and ENT3 strain.

FIG. 10 shows the gut macroscopical damage scores for mice treated with the negative controls PBS or Baker's yeast, with the SDH1 strain, ENT strain and ENT3 strain.

FIGS. 11A and 11B shows the expression level of ALD4 at 8 h (FIG. 11A) and 24 h (FIG. 11B) growth in the ENT, ENT1, ENT2 and ENT3 strains. Strains were grown in YP supplemented with 2% glucose at 37° C. The comparative Ct analysis was performed with qBase+(Biogazelle). ACT1, 18s, SCR1 were selected as stable reference genes for samples harvested at 8 h, and 18s, SCR1 for samples of 24 h. Normalized relative quantities (NRQ) were scaled to the parent ENT strain. Statistical analysis was done using Ordinary One-way ANOVA (Tukey's multiple comparisons test, *p≤0.05, **p≤0.01, ***p≤0.001, ****p≤0.0001, ns—non significant). Data represents mean values±SD from three independent colonies.

FIG. 12 shows the comparison between the viability of S. boulardii strains SbP, ENT and ENT3 after exposure to simulated gastric acid of pH 1.7 (black bars) and pH 1.2 (gray bars). Tolerance to the low pH of simulated gastric acid was determined by comparing CFU of cells exposed to saline (NaCl 0.5%) at pH 6.8 to pH 1.7 or 1.2 respectively. Cells were incubated for 3 h at 37° C. (*p≤0.05, ***p≤0.001. All groups compared to each other using one way ANOVA)

FIG. 13 shows the effect of ENT3 modifications (i.e. ach1Δ ALD4-OE) in two other wild type S. boulardii strains UL and 7103. Cells were propagated in YPD2% at 37° C., 200 rpm for 72 h.

### DETAILED DESCRIPTION

### Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Michael R. Green and Joseph Sambrook, Molecular Cloning: A Laboratory Manual, 4^{th} ed., Cold Spring Harbor Laboratory Press, Plainsview, New York (2012); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

In this application, engineered S. boulardii yeasts are reported with superior probiotic characteristics.

The inventors have demonstrated that the engineered S. boulardii strains are able to accumulate much higher levels of the antibacterial compound acetic acid compared to currently available S. boulardii strains while being resistant to acidic environments. The increased acetic acid production is attributed to a reduced activity of the tricarboxylic acid (TCA) cycle and/or by compromising Cit1 and/or Ach1 function. The acetic acid production can be further enhanced by overexpression of ALD4. The resulting engineered strains are herein shown to be able to accumulate acetic acid when growing on glucose levels as low as 20 mM, such as those present in the gastrointestinal tract between meals. Moreover, the inventors have confirmed the antimicrobial capacity of acetic acid against potential bacterial pathogens isolated from the gut and demonstrated that the culture supernatant produced by the engineered S. boulardii stains of the invention has higher antibacterial capacity under both aerobic and anaerobic conditions compared to the currently available S. boulardii strains.

“Acetic acid” or “acetate” (systematically named ethanoic acid) as used herein refers to the colorless liquid organic compound with the chemical formula CH₃COOH (also written as CH₃CO₂H or C₂H₄O₂). Acetic acid is the second simplest carboxylic acid (after formic acid). It consists of a methyl group attached to a carboxyl group. It is an important chemical reagent and industrial chemical, used primarily in the production of cellulose acetic acid for photographic film, polyvinyl acetic acid for wood glue, and synthetic fibres and fabrics. In households, diluted acetic acid is often used in descaling agents. In the food industry, acetic acid is controlled by the food additive code E260 as an acidity regulator and as a condiment. As a food additive it is approved for usage in many countries. Acetic acid is also known as an antibiotic compound, as was demonstrated in this application, e.g. Example 6. Also in the art, extensive evidence for acetic acid as anti-microbial compound is available (e.g. Rhee et al 2003 Appl Environ Microbiol 69: 2959-2963; Ryssel et al 2009 Burns 35: 695-700; Fraise et al 2013 J Hosp Infec 84: 329-331).

As introduced earlier, Saccharomyces boulardii (or also known as S. cerevisiae var. boulardii) is a well-known probiotic. It is administered to humans and animals with the purpose of introducing beneficial active cultures into the large and small intestine, as well as to confer protection against pathogenic (gut) microorganisms. Many S. boulardii strains are available including several strains that are commercially available. Of particular interest for this application is S. boulardii strain ENT or CMCN 1-745. These names are used interchangeable herein. The ENT or CMCN 1-745 strain is described in Kazmierczak-Siedlecka et al '2020 Curr Microbiol 77) and in Offei et al (2019 Genome Res 29), which are herein incorporated as reference.

It is demonstrated herein that knocking-out CIT1 or ACH1 gene expression in S. boulardii yeast comprising at least one wild-type WHI2 allele (e.g. ENT strain) significantly increases the production of acetate. Moreover, instead of the common transient acetate production that is observed when modulating acetate production in S. boulardii, it was unexpectedly observed that CIT1 or ACH1 knock-out resulted in a permanently high acetate production (see FIG. 2B in Example 2).

CIT1 and/or ACH1 Disruption in S. boulardii

“CIT1” or “Cit1” as used herein refers to the CITRATE SYNTHASE 1 gene or Citrate synthase 1 protein, respectively, of Saccharomyces. CIT1 also known as YNR001C (SGD ID: S000005284; Chromosome XIV 629622 . . . 631061), CS1 and LYS6, encodes a mitochondrial citrate synthase that catalyzes the condensation of acetyl coenzyme A and oxaloacetate to form citrate, which is the first and rate-limiting step of the TCA cycle. CIT1 has a paralog, CIT2, that arose from the whole genome duplication. The CIT1 gene is depicted in SEQ ID No. 1 and the Cit1 protein is depicted in SEQ ID No. 2.

“ACH1” or “Ach1” as used herein refers to the ACETYL COA HYDROLASE 1 gene or Acetyl-CoA hydrolase 1 protein, respectively, of Saccharomyces. ACH1 also known as YBL015W (SGD ID:S000000111; Chromosome II 194122 . . . 195702) encodes a protein with CoA transferase activity, particularly for CoASH transfer from succinyl-CoA to acetate. The ACH1 gene is depicted in SEQ ID No. 3 and the Ach1 protein is depicted in SEQ ID No. 4.

Knocking-out the expression of CIT1 and/or ACH1 results in an inhibition of the tricarboxylic acid (TCA) pathway. The TCA cycle, also known as the Krebs or citric acid cycle, is the main source of energy for cells and an important part of aerobic respiration. The cycle harnesses the available chemical energy of acetyl coenzyme A (acetyl CoA) into the reducing power of nicotinamide adenine dinucleotide (NADH).

Therefore and in a first aspect, a Saccharomyces boulardii yeast is provided in which the tricarboxylic acid pathway is compromised or partially abolished. This is equivalent as saying that a S. boulardii yeast is provided in which the activity of the TCA pathway is significantly reduced. This significant reduction can be an at least 10%, 15%, 20%, 25%, 30%, 35%, 40%, 45%, 50%, 60%, 70%, 80%, 90% or 95% reduction.

Said reduction of TCA activity can easily be measured by the skilled one, for example and without the purpose of being limited by the consumption of oxygen. In one embodiment, the TCA pathway is compromised or partially abolished because of the presence in the S. boulardii yeast of a disrupted, partially deleted or completely deleted CIT1 and/or ACH1 allele. Given that S. boulardii is diploid this is equivalent as saying that a S. boulardii yeast is provided in which both endogenous CIT1 and/or ACH1 alleles have been disrupted or deleted. A disrupted, partially deleted or completely deleted CIT1 and/or ACH1 allele is equivalent to an allele that compromises, partially abolishes or completely abolishes Cit1 or Ach1 function. In a particular embodiment, a S. boulardii yeast is provided comprising a homozygous or hemizygous mutant CIT1 or ACH1 allele, wherein said mutant allele compromises, partially abolishes or completely abolishes Cit1 or Ach1 function.

The mutant CIT1 allele which is disclosed in this application is a loss-of-function deletion. However, any CIT1 mutation that would result in an inactive Cit1 protein would have the same effect. Non-limiting examples of such CIT1 mutant alleles are alleles containing one or more non-synonymous point mutations in the open reading frame (ORF), or one or more mutations in the promoter and/or terminator sequence, causing compromised, partially abolished or completely abolished Cit1 function.

The same hold true for the ACH1 mutant allele. While a loss-of-function deletion was used in the Examples, the nature of the mutation is not important as long as the mutation results in an inactive Ach1 protein. Non-limiting examples of ACH1 mutant alleles are alleles containing one or more non-synonymous point mutations in the ORF, or one or more mutations in the promoter and/or terminator sequence, causing compromised, partially abolished or completely abolished Ach1 function.

“Homozygous” refers to having identical alleles for a single trait. An “allele” represents one particular form of a gene. Alleles can exist in different forms and diploid organisms typically have two alleles for a given trait. A homozygous mutant CIT1 or ACH1 allele thus means that all CIT1 or ACH1 alleles are identical. “Hemizygous” refers to having only one allele for a single trait or gene. In case of a diploid organism thus only one allele of its pair is present, while all other genes are represented by two alleles.

This can for example be achieved by deleting one allele of a gene or by introducing one allele of a gene that is not present in an organism.

“Disrupted, partially deleted or completely deleted function” or “disrupting, partially deleting or completely deleting the functional expression” is equivalent to partially or completely inhibiting the formation of a functional mRNA molecule encoding Cit1 or Ach1.

In another embodiment, an engineered S. boulardii yeast is provided having a statistically significantly reduced expression of CIT1 and/or ACH1 compared to the non-engineered S. boulardii control yeast. In yet another embodiment, said statistically significantly reduced expression of CIT1 and/or ACH1 is an at least 25%, 40%, 50%, 60%, 70%, 80%, 85%, 90%, 95%, 96%, 97%, 98%, 99% or 100% reduction of the CIT1 and/or ACH1 expression from an non-engineered control S. boulardii. The skilled person is familiar how to select a proper control S. boulardii yeast. A control yeast should have the same genetic background except for the parameter (e.g. CIT1 and/or ACH1 expression) that is to be tested.

It will be understood that methods for gene disruption or deletion in yeast and other microorganisms are well known, and (as earlier stated) the particular method used to reduce or abolish the expression of the endogenous gene is not critical to the invention. Disruption of an allele as used herein means inserting a DNA fragment in the base sequence of said allele or deleting or mutating a portion of said allele so that the allele cannot function any longer. Said inserted DNA fragment or said deleted or mutated portion can be as small as 1 base and as long as a plasmid or fragment thereof. As a result of gene (or allele) disruption, the gene (or allele) cannot be transcribed into mRNA, hence the structural gene is not translated, or the transcription product mRNA becomes incomplete, hence mutation or deletion occurs in the amino acid sequence of the translation product structural protein, rendering the protein incapable of performing the original function. In order to disrupt the CIT1 and/or ACH1 allele, any site may be disrupted, for example, a promoter site of CIT1 and/or ACH1, an open reading frame (ORF) site, and a terminator site, or combination thereof may be disrupted.

Methods for gene knockout and multiple gene knockout are well known. See e.g. Rothstein 2004, “Targeting, Disruption, Replacement, and Allele Rescue: Integrative DNA Transformation in Yeast” In: Guthrie et al., Eds. Guide to Yeast Genetics and Molecular and Cell Biology, Part A, p. 281-301; Wach et al., 1994, “New heterologous modules for classical or PCR-based gene disruptions in Saccharomyces cerevisiae” Yeast 10:1793-1808. Methods for insertional mutagenesis are also well known. See, e.g., Amberg et al., eds., 2005, Methods in Yeast Genetics, p. 95-100; Fickers et al., 2003, “New disruption cassettes for rapid gene disruption and marker rescue in the yeast Yarrowia lipolytica” Journal of Microbiological Methods 55:727-737; Akada et al., 2006, “PCR-mediated seamless gene deletion and marker recycling in Saccharomyces cerevisiae” Yeast 23:399-405; Fonzi et al., 1993, “Isogenic strain construction and gene mapping in Candida albicans” Genetics 134:717-728.

In one embodiment, disruption can be accomplished by homologous recombination, whereby the CIT1 and/or ACH1 gene is interrupted (e.g. by the insertion of a selectable marker gene) or made inoperative (e.g. “gene knockout”) after transforming a plasmid or a fragment thereof for disrupting the CIT1 and/or ACH1 allele into S. boulardii. In case that a plasmid for disruption of the CIT1 and/or ACH1 gene or a fragment thereof and the CIT1 and/or ACH1 gene on the S. boulardii genome have a homology to an extent for causing homologous recombination, homologous recombination is caused.

In another embodiment, disruption can be accomplished by random or site-directed mutagenesis. Random mutagenesis introduces mutations in a random fashion, e.g. upon treating yeast with radiation or chemical mutagens. Site-directed mutagenesis is a molecular biology method that is used to make specific and intentional changes to the DNA sequence of a gene and any gene products, e.g. specifically in CIT1 or ACH1. Non-limiting examples of mutations are point mutations, nonsense mutations, missense mutations, frameshift mutations, knock-out mutations or loss-of-function mutations. But also gain-of-function mutations and dominant negative mutations can disrupt the functional expression or inhibit the formation of a functional mRNA molecule. A “knock-out” can be a gene knockdown (leading to reduced gene expression) or the gene can be knocked out by a mutation such as a point mutation, an insertion, a deletion, a frameshift or a missense mutation by techniques known in the art. The lack of transcription can e.g. be caused by epigenetic changes (e.g. DNA methylation) or by loss-of-function mutations. A “loss-of-function” or “LOF” mutation as used herein is a mutation that prevents, reduces or abolishes the function of a gene product as opposed to a gain-of-function mutation that confers enhanced or new activity on a protein. The deletion or loss-of-function mutation in the CIT1 or ACH1 allele has a loss-of-function effect and is recessive, meaning that the deletion or mutation has to be homozygous or hemizygous to lead to the mutant phenotype. Both dominant negative or LOF mutations can be caused by a wide range of mutation types, including, but not limited to, a deletion of the entire gene or part of the gene, splice site mutations, frame-shift mutations caused by small insertions and deletions, nonsense mutations, missense mutations replacing an essential amino acid and mutations preventing correct cellular localization of the product. A “nonsense mutation” as used herein refers to a point mutation in a sequence of DNA that results in a premature stop codon (often illustrated as *), or a nonsense codon in the transcribed mRNA, and in a truncated, incomplete, and nonfunctional protein product. A “missense mutation” means a point mutation where a single nucleotide is changed to cause substitution of a different amino acid.

TCA can also be inhibited or Cit1 and/or Ach1 function can be abolished by deleting the whole CIT1 and/or ACH1 gene. Therefore in alternative embodiments, a S. boulardii yeast is provided comprising a completely deleted CIT1 and/or ACH1 allele or a S. boulardii yeast is provided devoid of the CIT1 and/or ACH1 allele or deficient of the CIT1 and/or ACH1 allele. In particular embodiments, said S. boulardii yeasts comprise a homozygous or hemizygous disrupted CIT1 and/or ACH1 allele. Also, a S. boulardii yeast is provided in which the CIT1 and/or ACH1 allele has been disrupted or deleted by homologous recombination. In another embodiment, an S. boulardii yeast is provided in which all CIT1 and/or ACH1 alleles have been deleted.

Other methods to disrupt a gene in a microorganism include the use of nucleases, such as zinc-finger nucleases (ZFNs), Transcription Activator-Like Effector Nucleases (TALENs), meganucleases but especially the CRISPR-Cas system. “Nucleases” as used herein are enzymes that cut nucleotide sequences. These nucleotide sequences can be DNA or RNA. If the nuclease cleaves DNA, the nuclease is also called a DNase. If the nuclease cuts RNA, the nuclease is also called an RNase. Upon cleavage of a DNA sequence by nuclease activity, the DNA repair system of the cell will be activated. Yet, in most cases the targeted DNA sequence will not be repaired as it originally was and small deletions, insertions or replacements of nucleic acids will occur, mostly resulting in a mutant DNA sequence. ZFN are artificial restriction enzymes generated by fusing a zinc finger DNA-binding domain to a DNA cleavage domain. Zinc finger domains can be engineered to target desired DNA sequences, which enables zinc-finger nucleases to target a unique sequence within a complex genome. By taking advantage of endogenous DNA repair machinery, these reagents can be used to precisely alter the genomes of simple and higher organisms. Other technologies for genome customization that can be used to knock out genes are meganucleases and TAL effector nucleases (TALENs, Cellectis bioresearch). A TALEN is composed of a TALE DNA binding domain for sequence-specific recognition fused to the catalytic domain of an endonuclease that introduces double strand breaks (DSB). The DNA binding domain of a TALEN is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). Another recent and very popular genome editing technology is the CRISPR-Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway and has been modified to edit basically any genome. By delivering the Cas nuclease (in many cases Cas9) complexed with a synthetic guide RNA (gRNA) in a cell, the cell's genome can be cut at a desired location depending on the sequence of the gRNA, allowing existing genes to be removed and/or new one added and/or more subtly removing, replacing or inserting single nucleotides (e.g. DiCarlo et al 2013 Nucl Acids Res doi:10.1093/nar/gkt135; Sander & Joung 2014 Nat Biotech 32:347-355). Therefore, also an S. boulardii yeast is provided in which the CIT1 and/or ACH1 allele has been disrupted or deleted by using nuclease technology, more particularly by means of the CRISPR-Cas technology.

Whether the CIT1 or ACH1 allele is disrupted or deleted in S. boulardii by any of the technologies available to the skilled including but not limited to those explained herein above, can be confirmed by quantifying the expression level of CIT1 or ACH1 or alternatively by growing the S. boulardii yeast and measuring the acetic acid produced. Disruption or deletion of CIT1 or ACH1 can be confirmed when the S. boulardii produces a statistically higher acetic acid level compared to a control S. boulardii, more particularly, when producing at least 5.8 g/l, at least 6 g/l, at least 6.2 g/l, at least 6.4 g/l, at least 6.6 g/l, at least 6.8 g/l, at least 7 g/l, at least 7.2 g/l, at least 7.4 g/l, at least 7.6 g/l, at least 7.8 g/l or at least 8 g/l acetate for 72 h at 37° C. in the presence of 110 mM glucose or in YPD2%. Alternatively confirmation of CIT1 and/or ACH1 disruption can be obtained by measuring the pH of the supernatant, more particularly when the supernatant of the yeast culture acidifies to a pH lower than 5, preferably lower than 4.8, more preferably lower than 4.4, lower than 4.2, lower than 4 or lower than 3.8. All S. boulardii yeasts described above are from here on referred to as the S. boulardii yeasts of the first aspect of the invention.

Overexpression of ALD4 and/or ALD6

The inventors of current application also demonstrated that increasing the expression of ALD4 or ALD6 in S. boulardii yeasts increased the acetic acid production. Hence, in a second aspect, a S. boulardii yeast is provided with a statistically significantly enhanced ALD4 and/or ALD6 expression compared to a wild-type control S. boulardii yeast.

“ALD4” or “Ald4” as used herein refers to the ALDEHYDE DEHYDROGENASE 4 gene or Aldehyde dehydrogenase 4 protein, respectively, of Saccharomyces. ALD4 is also known as YOR374W (SGD ID: 5000005901; Chromosome XV 1039840 . . . 1041399), ALDH2 and ALD7. Ald4 is a mitochondrial aldehyde dehydrogenase required for growth on ethanol and conversion of acetaldehyde to acetate. In one embodiment, ALD4 as used herein refers to the gene encoding Ald4 protein as depicted in SEQ ID No 6. In a particular embodiment, ALD4 as used herein is the gene as depicted in SEQ ID No. 5.

“ALD6” or “Ald6” as used herein refers to the ALDEHYDE DEHYDROGENASE 6 gene or Aldehyde dehydrogenase 6 protein respectively of Saccharomyces. ALD6 is also known as YPL061W (SGD ID: S000005982; Chromosome XVI 432588 . . . 434090) and ALD1. Ald6 is a cytosolic aldehyde dehydrogenase required for conversion of acetaldehyde to acetate. In one embodiment, ALD6 as used herein refers to the gene encoding Ald6 protein as depicted in SEQ ID No 8. In a particular embodiment, ALD6 as used herein is the gene as depicted in SEQ ID No. 7.

By “encoding” or “encodes” or “encoded”, with respect to a specified nucleic acid, is meant comprising the information for transcription into an RNA molecule and in some embodiments, translation into the specified protein or amino acid sequence. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

Enhancing or increasing the expression of one of more genes in yeast can be achieved by multiple approaches known by the skilled person in the art. In one embodiment, the ALD4 and/or ALD6 expression is enhanced by transforming the yeast with a chimeric gene construct comprising at least one ALD4 and/or ALD6 allele.

A “chimeric gene” or “chimeric construct” or “chimeric gene construct” as used herein is a recombinant nucleic acid sequence in which a promoter or regulatory nucleic acid sequence is operably linked to, or associated with, a nucleic acid sequence that codes for a mRNA (e.g. ALD4) and encodes an amino acid sequence (e.g. Ald4), such that the regulatory nucleic acid sequence is able to regulate transcription or expression of the associated nucleic acid coding sequence. The regulatory nucleic acid sequence of the chimeric gene is not operably linked to the associated nucleic acid sequence as found in nature. A chimeric gene construct can also comprise a 3′ end region involved in transcription termination or polyadenylation.

A “promoter” comprises regulatory elements, which mediate the expression of a nucleic acid molecule. For expression, the nucleic acid molecule must be linked operably to or comprise a suitable promoter which expresses the gene at the right point in time and with the required spatial expression pattern. The term “operably linked” as used herein refers to a functional linkage between the promoter sequence and the gene of interest, such that the promoter sequence is able to initiate transcription of the gene of interest. A promoter that enables the initiation of gene transcription in a eukaryotic cell is referred to as being “active”. To identify a promoter which is active in a eukaryotic cell, the promoter can be operably linked to a reporter gene after which the expression level and pattern of the reporter gene can be assayed. Suitable well-known reporter genes include for example beta-glucuronidase, beta-galactosidase or any fluorescent or luminescent protein. The promoter activity is assayed by measuring the enzymatic activity of the beta-glucuronidase or beta-galactosidase. Alternatively, promoter strength may also be assayed by quantifying mRNA levels or by comparing mRNA levels of the nucleic acid, with mRNA levels of housekeeping genes such as 18S rRNA, using methods known in the art, such as Northern blotting with densitometric analysis of autoradiograms, quantitative real-time PCR or RT-PCR (Heid et al. 1996 Genome Methods 6: 986-994).

The term “a 3′ end region involved in transcription termination or polyadenylation” encompasses a control sequence which is a DNA sequence at the end of a transcriptional unit which signals 3′ processing or polyadenylation of a primary transcript and is involved in termination of transcription. The control sequence for transcription termination or terminator can be derived from a natural gene or from a variety of genes. For expression in yeast the terminator to be added may be derived from, for example, the TEF or CYC1 genes or alternatively from another yeast gene or less preferably from any other eukaryotic or viral gene.

In a particular embodiment, a chimeric gene construct is provided comprising a promoter which is active in S. boulardii, a nucleic acid molecule encoding the Ald4 and/or Ald6 protein and a 3′ end region involved in transcription termination and/or polyadenylation. In a further particular embodiment, said Ald4 protein is as depicted in SEQ ID No. 6. In another further particular embodiment, said nucleic acid molecule encoding the Ald4 protein is as depicted in SEQ ID No. 5. In a further particular embodiment, said Ald6 protein is as depicted in SEQ ID No. 8. In another further particular embodiment, said nucleic acid molecule encoding the Ald6 protein is as depicted in SEQ ID No. 7.

In a particular embodiment the promoter in the chimeric gene herein described is active in S. boulardii. and selected from the list comprising pTEF1 (Translation Elongation Factor 1); pTEF2; pHXT1 (Hexose Transporter 1); pHXT2; pHXT3; pHXT4; pTDH3 (Triose-phosphate Dehydrogenase) also known in the art as pGADPH (Glyceraldehyde-3-phosphate dehydrogenase) or pGDP or pGLD1 or pHSP35 or pHSP36 or pSSS2; pTDH2 also known in the art as pGLD2; pTDH1 also known in the art as pGLD3; pADH1 (Alcohol Dehydrogenase) also know in the art as pADC1; pADH2 also known in the art as pADR2; pADH3; pADH4 also known in the art as pZRG5 or pNRC465; pADH5; pADH6 also known in the art as pADHVI; pPGK1 (3-Phosphoglycerate Kinase); pGAL1 (Galactose metabolism); pGAL2; pGAL3; pGAL4; pGAL5 also known in the art as pPGM2 (Phosphoglucomutase); pGAL6 also known in the art as pLAP3 (Leucine Aminopeptidase) or pBLH1 or pYCP1; pGAL7; pGAL10; pGAL11 also known in the art as pMED15 or pRAR3 or pSDS4 or SPT13 or ABE1; pGAL80; pGAL81; pGAL83 also know in the art as pSPM1; pSIP2 (SNF1-interacting Protein) also know in the art as pSPM2; pMET (Methionine requiring); pPMA1 (Plasma Membrane ATPase) also known in the art as pKT110; pPMA2; pPYK1 (Pyruvate Kinase) also known in the art as pCDC19; pPYK2; pENO1 (Enolase) also known in the art as pHSP48; pENO2; pPHO (Phosphate metabolism); pCUP1 (Cuprum); pCUP2 also known in the art as pACE1; pPET56 also known in the art as pMRM1 (Mitochondrial rRNA Methyltransferase); pNMT1 (N-Myristoyl Transferase) also known in the art as pCDC72; pGRE1 (Genes de Respuesta a Estres); pGRE2; GRE3; pSIP18 (Salt Induced Protein); pSV40 (Simian Vacuolating virus) and pCaMV (Cauliflower Mosaic Virus). These promoters are widely used in the art. The skilled person will have no difficulty identifying them in databases. For example, the skilled person will consult the Saccharomyces genome database website (http://www.yeastgenome.org/) or the Promoter Database of Saccharomyces cerevisiae (http://rulai.cshl.edu/SCPD/) for retrieving the yeast promoters' sequences. In a particular embodiment, the promoter is the TEF1 promoter.

Also a vector is provided comprising the above described chimeric gene construct. The term “vector” refers to any linear or circular DNA construct comprising the above described chimeric gene. The vector can refer to an expression cassette or any recombinant expression system for the purpose of expressing ALD4 and/or ALD6 in S. boulardii, constitutively or inducibly. The vector can remain episomal or integrate into the host cell genome. The vector can have the ability to self-replicate or not (i.e. drive only transient expression in a cell). In one embodiment, the vector is a “recombinant vector” which is by definition a man-made vector.

In one embodiment, an S. boulardii yeast, particularly an engineered S. boulardii yeast, is provided wherein the expression of ALD4 and/or ALD6 is increased by at least 25%, 50%, 60%, 70%, 80%, 90%, 100% or at least 2-fold, 3-fold, 5-fold, or 10-fold compared to an S. boulardii not engineered for enhanced ALD4 and/or ALD6 expression or not comprising a chimeric gene construct comprising at least one ALD4 and/or ALD6 allele.

In another embodiment, an S. boulardii yeast is provided comprising any of the above chimeric gene constructs. In a particular embodiment, an S. boulardii yeasts is provided comprising at least two, at least three, at least four, at least five, at least six, at least 8 or at least 10 chimeric gene constructs comprising at least one ALD4 and/or ALD6 allele. Alternatively, the S. boulardii yeast can also comprise a chimeric gene construct comprising at least two, at least three, at least four, at least five, at least six, at least 8 or at least 10 copies of the ALD4 and/or ALD6 allele.

Optimizing Acetic Acid Production in S. boulardii

The inventors demonstrated that the observed permanent acetate production in the S. boulardii yeasts having a compromised, partially deleted or completely Act1 or Cit1 function (described in the first aspect of the invention) could be additionally enhanced by overexpressing ALD4. Hence, in a third aspect, any of the above described S. boulardii yeasts having a compromised, partially deleted or completely deleted Cit1 and/or Ach1 function or any of the S. boulardii yeasts described in the first aspect of the invention is provided additionally having a statistically significantly enhanced expression of ALD4 compared to a control S. boulardii yeast. Said control yeast is an S. boulardii having a compromised, partially deleted or completely deleted Cit1 and/or Ach1 function but not engineered for enhanced ALD4 and/or ALD6 expression.

In a particular embodiment, any of the S. boulardii yeasts from the first aspect of the invention is provided further comprising at least one, at least two, at least three, at least four, at least five, at least six, at least 8 or at least 10 chimeric gene constructs comprising at least one ALD4 allele. Alternatively, the S. boulardii yeast can also comprise a chimeric gene construct comprising at least one, at least two, at least three, at least four, at least five, at least six, at least 8 or at least 10 copies of the ALD4 allele. Expressing additional copies of the ALD4 gene in the yeasts described in the first aspect of the invention not only further increases the acetic acid production, it also allows the S. boulardii yeast to produce acetic acid at glucose concentration as low as 20 mM, a condition in which no other S. boulardii strain is able to produce acetic acid.

In another embodiment, any of the S. boulardii yeasts according to the first aspect of the invention is provided wherein the expression of ALD4 is increased by at least 25%, 50%, 60%, 70%, 80%, 90%, 100% or at least 2-fold, 2.25-fold, 2.5-fold, 2.8-fold, 3-fold, 5-fold, or at least 10-fold compared to an S. boulardii not engineered for enhanced ALD4 expression or not comprising a chimeric gene construct comprising at least one ALD4 allele during the exponential growth phase of the yeast and/or wherein the expression of ALD4 is increased by at least 25%, 50%, 60%, 70%, 80%, 90%, 100% or at least 1.2-fold, 1.3-fold, 1.4-fold, 1.5-fold, 1.6-fold, 1.7-fold, 1.8-fold, 1.9-fold, or at least 2-fold compared to an S. boulardii not engineered for enhanced ALD4 expression or not comprising a chimeric gene construct comprising at least one ALD4 allele during the stationary or early stationary growth phase of the yeast. In a further embodiment, said S. boulardii yeasts comprise at least one, at least two, at least three, at least four, at least five, at least six, at least 8 or at least 10 chimeric gene constructs comprising at least one ALD4 allele or a chimeric gene construct comprising at least one, at least two, at least three, at least four, at least five, at least six, at least 8 or at least 10 copies of the ALD4 allele.

In a fourth aspect, a Saccharomyces boulardii yeast is provided producing acetic acid at 37° C. in low glucose medium of about 20 mM glucose. In one embodiment, said yeast produces at least 1 g/l acetic acid. In another embodiment, said yeast is one of the S. boulardii yeasts according to the first aspect of the invention additionally overexpressing ALD4 by at least 50%, at least 75%, at least 100%, at least 1.5-fold, at least 2-fold, at least 2.25-fold, at least 2.5-fold, at least 2.8-fold or at least 3-fold compared to a wild-type control strain and/or comprising at least two chimeric gene constructs comprising at least one ALD4 allele and/or comprising at least one chimeric gene construct comprising at least two ALD4 allele copies. In one embodiment said at least two chimeric gene constructs are those described in current application, more particularly in the second aspect of the invention. In another embodiment, the level of ALD4 expression is determined at the exponential phase of growing the S. boulardii strain. In yet another embodiment, the level of ALD4 expression is determined at the stationary or early stationary growth phase. In another embodiment, the S. boulardii strain having increased ALD4 expression is an S. boulardii strain engineered for increased ALD4 expression and the wild-type control strain to which the ALD4 expression is compared to is a control or isogenic S. boulardii strain not engineered for increased ALD4 expression.

In a fifth aspect, a Saccharomyces boulardii yeast is provided producing at least 0.25 g/l, 0.5 g/l, 0.75 g/l, 1 g/l or at least 1.25 g/l acetic acid at 37° C. in low glucose medium of about 20 mM glucose. In one embodiment, the S. boulardii yeast permanently produces acetic acid at 37° C. in low glucose medium of about 50 mM glucose. In one embodiment, said yeast produces at least 1 g/l, 2 g/l or 3 g/l acetic acid. In another embodiment, the S. boulardii yeast permanently produces at least 1 g/l, 2 g/l, 3 g/l, 4 g/l, 5 g/l or at least 6 g/l acetic acid at 37° C. in a medium of about 75 mM glucose. In another embodiment, said yeast is one of the S. boulardii yeasts according to the first aspect of the invention additionally overexpressing ALD4 by at least 50%, at least 75%, at least 100%, at least 2-fold, at least 2.5-fold, at least 3-fold, at least 4-fold, at least 5-fold, at least 6-fold, at least 8-fold or at least 10-fold compared to a wild-type control strain and/or comprising at least four chimeric gene constructs comprising at least one ALD4 allele and/or comprising at least one chimeric gene construct comprising at least four ALD4 allele copies. In one embodiment said at least two chimeric gene constructs are those described in current application, more particularly in the second aspect of the invention.

In a sixth aspect, a Saccharomyces boulardii yeast is provided producing a cell-free supernatant with a pH lower than 5 in a temperature range between 35° C. and 39° C., or between 36° C. and 38° C., or between 36° and 37.5°, or between 36.5° C. and 37.5° C. or most particularly at 37° C., wherein the acidification of said supernatant is due to the production or accumulation of acetic acid by said S. boulardii yeast and wherein said S. boulardii yeast is any of the S. boulardii yeasts described above or herein. In particular embodiments, said “pH lower than 5” is a pH lower than 4.9, or lower than 4.8, or lower than 4.7, or lower than 4.6, or lower than 4.5, or lower than 4.4, or lower than 4.3, or lower than 4.2, or lower than 4.1, or lower than 4, or lower than 3.9, or lower than 3.8 or lower than or equal to 3.6 or is a pH between 5 and 4.6, or between 4.9 and 4.2, or between 4.8 and 4.1, or between 4.6 and 4 or between 4.4 and 3.8.

In a seventh aspect, an enriched culture of any of the S. boulardii yeasts herein described is provided. The term “culture” as used herein refers to a population of microorganisms that are propagated on or in media of various kinds. An “enriched culture” of any of the S. boulardii yeasts of current application refers to a culture of microorganisms, more particular a yeast culture, wherein the total microbial population of the culture contains more than 50%, more than 60%, more than 70%, more than 80%, more than 90%, or more than 95% of any of the S. boulardii yeasts of current application. This is equivalent as saying that a culture of microorganisms, more particularly a yeast culture, is provided, wherein said culture is enriched with any of the S. boulardii yeasts of current application and wherein “enriched” means that the total microbial (or more particularly the total yeast) population of said culture contains more than 50%, more than 60%, more than 70%, more than 80%, more than 90%, or more than 95% of any of the S. boulardii yeasts of current application.

In one embodiment, a biologically pure culture of any of the S. boulardii yeasts of current application is provided. As used herein, “biologically pure” refers to a culture which contains substantially no other microorganisms than the desired strain of microorganism and thus a culture wherein virtually all of the cells present are of the selected yeast or strain. In practice, a culture is defined biologically pure if the culture contains at least more than 96%, at least more than 97%, at least more than 98% or at least more than 99% of any of the S. boulardii yeasts or stains of current application. When a biologically pure culture contains 100% of the desired microorganism a monoculture is reached. A monoculture thus only contains cells of the selected strain and is the most extreme form of a biologically pure culture.

The S. boulardii Yeast of the Invention

In one general embodiment of the invention, any of the S. boulardii yeasts described above and herein is an engineered or recombinant S. boulardii yeast. “Engineering” or “engineered” as used herein refers to genetic engineering, a technique whereby an organism's genome is modified using biotechnology. This includes but is not limited to the transfer of genes within and across species boundaries, deleting fragments of genes or deleting whole genes, modifying the DNA sequence of an organism by deleting, inserting or substituting one or more nucleic acid molecules. Means and methods to engineer microorganisms, particularly yeasts are well known by the person skilled in the art. The most known techniques involve traditional genetic transformation of yeast and recombinant DNA techniques. Nowadays, the most attractive technique to engineer a microorganism is by the use of nucleases, such as zinc-finger nucleases (ZFNs), Transcription Activator-Like Effector Nucleases (TALENs), meganucleases but especially the CRISPR-Cas system as described earlier.

In another general embodiment of the invention, any of the S. boulardii yeasts described above and herein further comprises at least one WHI2 wild-type allele. The is equivalent as saying that said S. boulardii yeasts comprise a heterozygous, hemizygous or homozygous wild-type WHI2 allele. A WHI2 wild-type allele is a nucleic acid molecule encoding a functional Whi2 protein. “WHI2” or “Whi2” as used herein refers to the WHISKEY2 gene or Whiskey2 protein, respectively, of Saccharomyces. WHI2 is also known in the art as YOR043W (SGD ID: S000005569, Chromosome XV 410870 . . . 412330). The functional WHI2 gene is depicted in SEQ ID No. 9 and the functional Whi2 protein is depicted in SEQ ID No. 10.

In another general embodiment of the invention, any of the S. boulardii yeasts described above and herein further comprises a homozygous or hemizygous SDH1 mutant allele, more particularly a loss-of-function SDH1 allele. “SDH1” or “Sdh1” as used herein refers to the SUCCINATE DEHYDROGENASE1 gene or Succinate dehydrogenase1 protein, respectively, of Saccharomyces. SDH1 is also known in the art as SDHA or YKL148C (SGD ID: S000001631, Chromosome XI 169207 . . . 171129). The SDH1 gene from S. cerevisiae is depicted in SEQ ID No. 11 and the Sdh1 protein is depicted in SEQ ID No. 12. In one further embodiment, the mutant SDH1 allele comprises a mutated nucleic acid at position 950 of the open reading frame sequence depicted in SEQ ID No. 11, wherein said mutation is a missense mutation resulting in a non-functional Sdh1 protein. “Position 950” as used herein refers to the nucleobase that is 949 positions removed downstream from the first nucleobase (i.e. adenosine) from the start codon. This position is indicated in SEQ ID No. xx by underlining. In another embodiment, said SHD1 mutant allele is the sdh1F317Y allele or encodes an Sdh1 protein comprising a F317Y mutation. In more particular embodiments, said SDH1 allele comprises a T950A mutation. In even more particular embodiments, said SDH1 mutant allele encodes the Sdh1 protein as depicted in SEQ ID No. 14 or is the nucleic acid molecular as depicted in SEQ ID No. 13.

In another general embodiment of the invention, any of the S. boulardii yeasts described above and herein is an S. boulardii strain. In a particular embodiment, said S. boulardii strain is not the Sb.P or Sb.A strain. In yet another particular embodiment, said S. boulardii strain is selected from the list consisting of 7136, UL, 259, 7135, Sb.L, SAN, 7103, FLO, ENT, CMCN 1-745, LSB or any S. boulardii strain derived thereof. In a most particular embodiment of the invention, said S. boulardii strain is S. boulardii strain CMCN 1-745 or a S. boulardii strain derived thereof. In another most particular embodiment of the invention, said S. boulardii strain is S. boulardii strain ENT or a S. boulardii strain derived thereof.

In another general embodiment of the invention, any of the S. boulardii yeasts or strains described above and herein is a haploid segregant. Haploid cells contain one set of chromosomes, while diploid cells contain two. A haploid segregant as used herein is equivalent to a haploid spore, the result of sporulation. Yeasts are eukaryotic, single-celled microorganisms classified as members of the fungus kingdom and like all fungi, yeast may have asexual and sexual reproductive cycles. The most common mode of vegetative growth in yeast is asexual reproduction by budding. Here, a small bud or daughter cell, is formed on the parent cell. The nucleus of the parent cell splits into a daughter nucleus and migrates into the daughter cell. The bud continues to grow until it separates from the parent cell, forming a new cell. This reproduction cycle is independent of the yeast's ploidy, thus both haploid and diploid yeast cells can duplicate as described above. Haploid cells have in general a lower fitness and they often die under high-stress conditions such as nutrient starvation, while under the same conditions, diploid cells can undergo sporulation, entering sexual reproduction (meiosis) and producing a variety of haploid spores or haploid segregants, which can go on to mate (conjugate), reforming the diploid. The budding yeast Saccharomyces cerevisiae reproduces by mitosis as diploid cells when nutrients are abundant, but when starved, this yeast undergoes meiosis to form haploid spores. Haploid cells may then reproduce asexually by mitosis. Importantly, S. boulardii is sporulation deficient (Edwards-Ingram et al 2007 Appl Environ Microbiol 73: 2458-2467) and thus does not have the ability to naturally form haploid spores or haploid segregants.

Medical Uses of the S. boulardii of the Invention

It is well established in the art that S. boulardii is efficacious against bacterial infections, inflammatory bowel diseases and other gastrointestinal disorders (e.g. Jawhara & Poulain 2007 Med Mycol 45: 691-700; Collier et al 2011 J Anim Sc 89: 52-58; Justino et al 2014 BrJ Nutr 111: 1611-1621; Martins et al 2013 Microbes and Infection 15: 270-279). In current application, the inventors demonstrate that at least a significant part of the antimicrobial activity of the herein disclosed S. boulardii yeasts can be attributed to its production of the antimicrobial compound acetic acid. Moreover, it is herein disclosed that a partially or completely disrupted CIT1 and/or ACH1 allele lead to enhanced production of acetic acid by S. boulardii yeasts comprising said mutant allele or being devoid of an CIT1 and/or ACH1 allele and that said S. boulardii yeasts have increased antimicrobial activity in vitro. Hence the optimized S. boulardii strains of the application are envisaged to be used as medicament, as probiotic and/or as dietary supplement. The effect of optimized S. boulardii strains can be easily tested in vivo, especially if it is already known that said strains have antimicrobial activity in vitro. A selection of an overwhelming number of papers can be found below.

Jawhara and Poulain (2007, Med Mycol 45: 691-700) analysed the effect of Saccharomyces boulardii on inflammation and intestinal colonization by Candida albicans in a mice model for colitis. Experimental details can be found therein, but briefly BALB/c mice were colonized with C. albicans by oral gavage with a 200 ml suspension of 10⁷ yeast cells. A 1.5% solution of DSS was administered in drinking water 1 h after C. albicans oral challenge, while 10⁷ cells of S. boulardii was inoculated daily by oral gavage for 1 week. Faeces were collected daily for 2 weeks and samples of the colon were taken for histological scoring and real-time PCR (RT-PCR) analysis of inflammatory cytokines and toll-like receptors (TLRs). Both the colony forming units (CFUs) of C. albicans and the inflammation were greatly reduced in mice receiving S. boulardii.

Justino et al (2014, B J Nutr 111: 1611-1621) treated a 5-fluorouracil (5-FU)-induced intestinal mucositis mice model with S. boulardii. Mice were divided into control, control+5-FU or 5-FU+S. boulardii (16×10⁹ CFUs/kg) treatment groups, and the jejunum and ileum were removed after killing of mice for the evaluation of histopathology and inflammation. S. boulardii significantly reversed the histopathological changes in intestinal mucositis induced by 5-FU and reduced the inflammatory parameters.

Martins et al (2013, Microbes and Infection 15: 270-279) challenged mice with Salmonella typhimurium (intragastrically with 0.1 ml of a bacterial suspension containing 10⁵ CFU/ml) with or without prior administration of S. boulardii. Treatment with S. boulardii (a daily dose of 0.1 ml containing 10⁹ CFU/ml by oral gavage starting 10 days before infection and continued throughout the experiment) increased survival rate and inhibited translocation of bacteria after S. typhimurium challenge. Histological data showed that S. boulardii also protected mice against liver damage induced by S. typhimurium. Additionally, S. boulardii decreased levels of inflammatory cytokines and signal pathways involved in the activation of inflammation induced by S. typhimurium.

Collier et al (2011, J Anim Sci 89: 52-58) tested the efficacy of S. boulardii to reduce mortality in pigs after an E. coli endotoxin challenge. Barrows were assigned to 1 of 2 treatment groups: with and without in-feed inclusion of S. boulardii (200 g/t) for 16 d. On d 16, all piglets were dosed via indwelling jugular catheters with LPS (25 ag/kg of BW) at 0 h. In S. boulardii-treated piglets, LPS-induced piglet mortality was reduced 20% compared with control piglets.

Pigs were also used by Daudelin et al (2011, Vet Res 42: 69). At birth, different litters of pigs were randomly assigned to a control group and to a S. boulardii group. S. boulardii was administered daily (1×10⁹ CFU per pig) during the lactation period and after weaning (day 21). At 28 days of age, all pigs were orally challenged with an ETEC F4 strain, and a necropsy was performed 24 h later. Attachment of ETEC F4 to the intestinal mucosa was significantly reduced in pigs treated with S. boulardii.

Line et al (1998 Poultry Science 77: 405-410) tested the effect of S. boulardii supplemented feed on Salmonella and Campylobacter population in broilers. Broiler chicks were given ad libitum access to a standard feed supplemented with no yeast (control), 1 g or 100 g dried S. boulardii/kg feed. All chicks except negative controls were challenged on day 4 with 3.2×10⁸ CFU S. typhimurium and 6.5×10⁸ CFU C. jejuni by oral gavage. After 3 wk, the broilers were euthanatized and ceca were aseptically removed and analyzed for Salmonella and Campylobacter. Frequency of Salmonella colonization was significantly (P<0.05) reduced due to yeast treatment. Campylobacter colonization however was not significantly affected.

For ethical, technical, regulatory, and cost reasons, in vitro methods are increasingly used as an alternative to in vivo experimentations. A non-limiting example is described in Fleury et al (2017 Appl Microbiol Biotechnol 101:2533-2547). The herein described in vitro model of the piglet colon, the PigutIVM, reproduces the main biotic and abiotic parameters of the piglet colon: temperature, pH, retention time, supply of ileal effluents, complex, and metabolically active microbiota and self-maintained anaerobiosis.

In another aspect, a composition comprising any of the S. boulardii yeasts of current application is provided. In one embodiment, any of said S. boulardii yeasts is lyophilized, freeze dried or in the form of a dry powder. The application also provides a composition comprising a supernatant obtained from a culture of any of the S. boulardii yeasts of current application. Said culture can be an enriched culture or a biologically pure culture of any of the S. boulardii yeasts of current application. “Supernatant” refers to the liquid broth remaining when cells grown in broth are removed by centrifugation, filtration, sedimentation or other means well known in the art. The application also provides a composition comprising an extract obtained from a culture of any of the S. boulardii yeasts of current application. Said culture can be an enriched culture or a biologically pure culture of any of the S. boulardii yeasts of current application. An “extract” as used herein refers to various forms of microbial products. These products are obtained by removing the cell walls and/or cell membranes of the microorganisms, a process also known as lysis, thereby obtaining one or more endogenous products of the cultured microorganisms. Non-limiting examples of such products are amino acids, peptides, enzymes, secondary metabolites, vitamins, minerals. Removing the cell walls and/or cell membranes of the cultured microorganisms can be obtained by several procedures which are well-known by the person skilled in the art. Non-limiting examples are addition of chemicals (e.g. sodium chloride) to a microbial culture, heating the microbial culture or induce lysis in a mechanical way. An extract can also be obtained by autolysis of the microorganisms.

In one embodiment, the composition can further comprise a preservative.

In a particular embodiment, said composition is a dietary supplement, a probiotic composition or a pharmaceutical composition.

In another aspect of the invention, any of the herein disclosed S. boulardii yeasts is provided for use as a medicament. Also the dietary supplement, the probiotic composition and pharmaceutical composition comprising any of said yeasts are provided for use as a medicament. In one embodiment, said yeast as well as said dietary supplement, the probiotic composition or pharmaceutical composition is provided for use in the treatment or prevention of gastrointestinal disorders, more particularly for use in the treatment or prevention of diarrhea, for use in the treatment of gastrointestinal inflammation, for use in reducing gastrointestinal discomfort, for use in increasing gastrointestinal comfort, for use in improving immune health and/or for use in relieving constipation. This is equivalent as saying that methods of treating or preventing said gastrointestinal disorders are provided, or of maintaining or improving the health of the gastrointestinal tract in a human or animal. Said method comprising the step of administering to said human or animal a dietary supplement, the probiotic composition or pharmaceutical composition comprising any of the S. boulardii yeasts described herein. The herein provided methods of treating or preventing gastrointestinal disorders can be alternatively phrased as methods of increasing the acetic acid concentration in the gastrointestinal tract or as method of reducing the abundance in the gut of acetic acid sensitive microorganisms, particularly gut bacteria.

In one embodiment, said treatment or prevention of gastrointestinal disorders (or alternatively phrased: improving or maintaining the health of the gastrointestinal tract) comprises reducing the number of pathogenic bacteria found in the faeces or in the gut of a human or animal. In more particular embodiments, said pathogenic bacteria are selected from the group consisting of Enterobacter, Klebsiella, Vibrio, Blastocystis, Clostridium, Citrobacter, Escherichia, Salmonella, Shigella and mixtures thereof.

The medical uses and medical methods of this application are both for treating and preventing gastrointestinal disorders. Indeed, administration of certain live probiotic yeasts can help restore optimal intestinal flora in animals such as cattle, especially after stressful situations such as transport to a feedlot (Gedek, B., “Probiotics in Animal Feeding-Effects on Performance and Animal Health,” Feed Magazine, November 1987) but regular administration of probiotics also increases nutrient absorption efficiency and helps control the proliferation of harmful microorganisms in the animals' digestive tract that could otherwise cause disease conditions adversely affecting rates of animal development and weight gain.

In particular embodiments, strain-release compositions may be provided in the compositions. For example, it may be favourable, particularly where the S. boulardii yeast is to be delivered to children, to initially provide the S. boulardii in a composition, e g. an oil suspension, within a drinking straw whereby the one or more strains are transferred to a liquid drawn through the straw, e.g. a juice or milk from a drink carton. Such straws housing probiotic strains are described, for example, in EP1224128B1 (Biogaia AB).

In other particular embodiments, the pharmaceutical composition includes a pharmaceutically acceptable carrier. Said pharmaceutically acceptable carrier is preferably an ingestible carrier. As used herein the term “pharmaceutically acceptable carrier” refers to a substance that does not produce an adverse, allergic or other problematic reaction when administered to an animal, preferably a human. It may include any and all solvents, dispersion media, coatings, isotonic and absorption delaying agents and the like. For human administration, preparations should meet sterility, pyrogenicity, general safety and purity standards as required by the biological standards of the FDA and EMA.

The probiotic S. boulardii of this application must arrive in large number in the gut in order to settle there, and must not be destroyed by stomach acid as it passes the stomach. Therefore, in particular embodiments, the dietary supplement, probiotic composition and pharmaceutical composition herein provided comprise a therapeutically effective amount of any of the S. boulardii yeasts of current application. In more particular embodiments, said therapeutically effective amount is an amount of more than 10⁶ CFU (colony forming units), or of more than 10⁷ CFU, or of more than 10⁸ CFU or of more than 10⁹ CFU of said yeast per gram or per ml of said supplement or composition, or comprises between 10⁵ and 10¹⁵ CFU, or between 10⁶ and 10¹² CFU, or between 10⁷ and 10¹¹ CFU, or between 10⁸ and 6×10¹⁰ CFU, or between 10⁹ and 2×10¹⁰ CFU of said yeast per gram or per ml of said supplement or composition. “CFU” or “cfu” as used herein refers to colony-forming unit. This unit is well-known by the person skilled in the art of microbiology (as well as the methodology how to determine the number of colony-forming units) and is used to estimate the number of viable bacteria or fungal cells in a sample. “Viable” is defined as the ability to multiply via binary fission under controlled conditions. Counting with colony-forming units requires culturing the microbes and counts only viable cells, in contrast with microscopic examination which counts all cells, living or dead.

In one embodiment of the invention, the composition, the pharmaceutical or probiotic composition, the dietary supplement or the medicament herein disclosed further comprises additional probiotic strains, such as, for example, bacterial probiotic strains; prokaryotes probiotics other than bacteria; or fungal strains, preferably yeast strains. In one embodiment, said additional probiotic strains are selected from those naturally present in the gut of the subject, preferably in the human gut, more preferably in the gut of healthy human subjects. Examples of bacterial probiotic strains that may be used in the present invention include, but are not limited to Lactobacillus, Lactococcus, Bifidobacterium, Veillonella, Desemzia, Coprococcus, Collinsella, Citrobacter, Turicibacter, Sutterella, Subdoligranulum, Streptococcus, Sporobacter, Sporacetigenium, Ruminococcus, Roseburia, Proteus, Propionobacterium, Leuconostoc, Weissella, Pediococcus, Streptococcus, Prevotella, Parabacteroides, Papillibacter, Oscillospira, Melissococcus, Dorea, Dialister, Clostridium, Cedecea, Catenibacterium, Butyrivibrio, Buttiauxella, Bulleidia, Bilophila, Bacteroides, Anaerovorax, Anaerostopes, Anaerofilum, Enterobacteriaceae, Fermicutes, Atopobium, Alistipes, Acinetobacter, Slackie, Shigella, Shewanella, Serratia, Mahella, Lachnospira, Klebsiella, Idiomarina, Fusobacterium, Faecalibacterium, Eubacterium, Enterococcus, Enterobacter, Eggerthella. Examples of prokaryote strains that may be used in the present invention include, but are not limited to Archaea, Firmicutes, Bacteroidetes (such as, for example, Allistipes, Bacteroides ovatus, Bacteroides splachnicus, Bacteroides stercoris, Parabacteroides, Prevotella ruminicola, Porphyromondaceae, and related genus), Proteobacteria, Betaproteobacteria (such as, for example, Aquabacterium and Burkholderia), Gammaproteobacteria (such as, for example, Xanthomonadaceae), Actinobacteria (such as, for example, Actinomycetaceae and Atopobium), Fusobacteria, Methanobacteria, Spirochaetes, Fibrobacters, Deferribacteres, Deinococcus, Thermus, Cyanobacteria, Methanobrevibacteria, Peptostreptococcus, Ruminococcus, Coprococcus, Subdolingranulum, Dorea, Bulleidia, Anaerofustis, Gemella, Roseburia, Dialister, Anaerotruncus, Staphylococcus, Micrococcus, Propionobacteria, Enterobacteriaceae, Faecalibacteria, Bacteroides, Parabacteroides, Prevotella, Eubacterium, Bacilli (such as, for example Lactobacillus salivans and related species, Aerococcus, Granulicatella, Streptococcus bovis and related genus and Streptococcus intermedius and related genus), Clostridium (such as, for example Eubacterium hallii, Eubacterium limosum and related genus) and Butyrivibrio. Examples of fungal probiotic strains, preferably yeast probiotic strains that may be used in the present invention include, but are not limited Ascomycetes, Zygomycetes and Deuteromycetes, preferably from the groups Aspergillus, Torulopsis, Zygosaccharomyces, Hansenula, Candida, Saccharomyces (including S. boulardii strains that are not subject of current application), Clavispora, Bretanomyces, Pichia, Amylomyces, Zygosaccharomyces, Endomycess, Hyphopichia, Zygosaccharomyces, Kluyveromyces, Mucor, Rhizopus, Yarrowia, Endomyces, Debaryomyces, and/or Penicillium.

In one embodiment of the invention, the composition, the pharmaceutical or probiotic composition, the dietary supplement or the medicament further comprises a prebiotic. Examples of prebiotics that may be used in the present invention include, but are not limited to inulin and inulin-type fructans, oligofructose, xylose, arabinose, arabinoxylan, ribose, galactose, rhamnose, cellobiose, fructose, lactose, salicin, sucrose, glucose, esculin, tween 80, trehalose, maltose, mannose, mellibiose, mucus or mucins, raffinose, fructooligosaccharides, galacto-oligosaccharides, amino acids, alcohols, and any combinations thereof. Other non-limiting examples of prebiotics include water-soluble cellulose derivatives, water-insoluble cellulose derivatives, unprocessed oatmeal, metamucil, all-bran, and any combinations thereof. Examples of water-soluble cellulose derivatives include, but are not limited to, methylcellulose, methyl ethyl cellulose, hydroxyethyl cellulose, ethyl hydroxyethyl cellulose, cationic hydroxyethyl cellulose, hydroxypropyl cellulose, hydroxyethyl methylcellulose, hydroxypropyl methylcellulose, and carboxymethyl cellulose.

Any of the herein disclosed S. boulardii yeasts or the composition, pharmaceutical or probiotic composition, or medicament of the invention may be administered by several routes of administration. Examples of adapted routes of administration include, but are not limited to oral administration, rectal administration, administration via esophagogastroduodenoscopy, administration via colonoscopy, administration using a nasogastric or orogastric tube and the like.

The composition, probiotic composition, prebiotic composition, dietary supplement, pharmaceutical composition or medicament may be in the form of a beverage, e.g. drinking yoghurt, juice or milk, or other form of nutritional composition. It may be suitable for human and/or animal consumption. For example the composition may be in the form of a food product such as a dairy products, dairy drinks, yoghurt, cheese, confectionary, nutritional snack bar, fruit or vegetable juice or concentrate thereof, powders, malt or soy or cereal based beverages, breakfast cereal such as muesli flakes, cereal and/or chocolate bars, spreads, flours, milk, smoothies, chocolate, gels, ice creams, reconstituted fruit products, muesli bars, sauces, dips, drinks including dairy and non-dairy based drinks, sports supplements including dairy and non-dairy based sports supplements. In one embodiment of the invention, the composition, pharmaceutical or probiotic composition, or medicament is in the form of a food additive, drink additive, dietary supplement, nutritional product, medical food or nutraceutical composition. It may also be in the form of a nutritional supplement. For example, any of the herein disclosed S. boulardii yeasts may be provided in a nutritional oil suspension, e.g. within a capsule, which will release the probiotic strains in the GI tract.

Any of the herein disclosed S. boulardii yeasts or the composition, dietary supplement, pharmaceutical or probiotic composition, or medicament herein disclosed may also be in a solid form such as a tablet, pill, powder, granules, troches, suppository, capsules, soft gelatin capsules, sugar-coated pills, orodispersin tablets, effervescent tablets or other solids. It may be a controlled-release formulation for release of any of the herein disclosed S. boulardii yeasts in the GI tract. In another embodiment, the composition, dietary supplement, pharmaceutical or probiotic composition or medicament of the application may be in a liquid form, such as a drinkable solution, liposomal composition or suspension, e.g. an oil suspension. Such a liquid composition may be provided within an encapsulating substance to provide a capsule or microcapsules, again for release of the one or more probiotic strains in the GI tract. In one embodiment, the pharmaceutical composition additionally includes a non-microbial therapeutic agent, e.g. a chemical drug entity or a therapeutic biologic, suitable for the prophylaxis and/or treatment of the undesired inflammatory activity of concern, pain or an associated condition. Non-limiting examples of said chemical drug entities are inhibitors of cyclooxygenase activity (aspirin, celecoxib, diclofenac, diflunisal, etodolac, ibuprofen, indomethacin, ketoprofen, ketorolac, meloxicam, nabumetone, naproxen, oxaprozin, piroxicam, salsalate, sulindac, tolmetin, among others) or corticosteroids (prednisone, dexamethasone, hydrocortisone, methylprednisolone, among others) or analgesics (acetaminophen, duloxetine, paracetamol, among others) or in any combination thereof. Non-limiting example of therapeutic biologics are TNF-alpha blockers, anti-IL17A monoclonal antibodies, anti-CD20 antibodies.

In one embodiment, the composition, dietary supplement, probiotic or pharmaceutical composition or medicament of the invention further comprises excipients, diluent and/or carriers selected with regard to the intended route of administration. Examples of excipients, diluent and/or carriers include, but are not limited to water, phosphate buffer saline, anaerobic phosphate buffer saline, sodium bicarbonate, juice, milk, yogurt, infant formula, dairy product, colouring agents, such as, for example, titane dioxide (E171), iron dioxide (E172) and brilliant black BN (E151); flavouring agents; thickeners, such as, for example, glycerol monostearate; sweeteners; coating agents, such as, for example, refined colza oil, soya oil, peanut oil, soya lecithin or fish gelatin; diluting agents, such as, for example, lactose, monohydrated lactose or starch; binding agents, such as, for example, povidone, pregelatinized starch, gums, saccharose, polyethylene glycol (PEG) 4000 or PEG 6000; disintegrating agents, such as, for example, microcrystalline cellulose or sodium carboxymethyl starch, such as, for example, sodium carboxymethyl starch type A; lubricant agents, such as, for example, magnesium stearate; flow agent, such as, for example, colloidal anhydrous silica, etc.

In yet another aspect of the invention, the use of any of the S. boulardii yeasts herein disclosed is provided as a live probiotic additive to a food or feed product. Also, the use of the dietary supplement comprising any of the herein described S. boulardii yeasts is provided for preparing a food supplement and/or a probiotic and/or a functional food and/or a nutraceutical and/or functional ingredients intended for human beings and/or for animals. Also, said dietary supplement is provided for preparing food compositions intended to improve gastrointestinal comfort and/or to improve intestinal flora. “Probiotic” as used herein refers to any consumable yeast, more particularly a S. boulardii yeast that provides health benefits for humans and animals when consumed. Probiotics are considered to be generally safe and help restore the balance of intestinal flora, keep it stable by positively changing the composition of the intestinal flora of humans and animals and/or positively affect the part of the immune system, which communicates with the intestinal wall. Through the production of metabolites, such as acetic acid, lactic acid and hydrogen peroxide, probiotic microorganisms, for example, deteriorate the living conditions of undesirable microorganisms in the gut. The presence of probiotic microorganisms in the gut improves the digestion function and can both be used in a therapeutic set-up for example to treat gastrointestinal disorders as diarrhea or in a preventive set-up for example to maintain a well-balanced gut microbiome and gastrointestinal comfort. A “probiotic additive” or equivalently “probiotic supplement” is a substance in any shape or form that contains probiotics. More specifically, a probiotic substance can be dry or liquid and comprises live probiotics embedded in a matrix of sugars, proteins and/or polysaccharides. Hence, it may be a food product on its own.

The term “food or feed product” is intended to encompass any consumable matter of either plant or animal origin or of synthetic sources that contain a body of nutrients such as a carbohydrate, protein, fat vitamin, mineral, etc. The product is intended for the consumption by humans or by animals, such as domesticated animals, for example cattle, horses, pigs, sheep, goats, and the like. Pets such as dogs, cats, rabbits, guinea pigs, mice, rats, birds (for example chickens or parrots), reptiles and fish (for example salmon, tilapia or goldfish) and crustaceans (for example shrimp). The food product may be liquid or solid. It may include but is not limited to a liquid fermented solution such as milk or yoghurt. The feed product may include but is not limited to pelleted feeds or pet feed for example a snack bar, crunchy treat, cereal bar, snack, biscuit, pet chew, pet food, and pelleted or flaked feed for aquatic animals.

“Functional food” as used herein is a food given an additional function (often one related to health-promotion or disease prevention) by adding new ingredients for example a probiotic or more of existing ingredients. A “nutraceutical” is a pharmaceutical-grade and standardized nutrient that provides medical or health benefits including the prevention and/or treatment of a disease. A “dietary supplement” is a non-nutrient chemical with a biologically beneficial effect. Supplements as generally understood include vitamins, minerals, fiber, fatty acids, or amino acids, among other substances.

### Methods of Producing Acetic Acid and Acetic Acid Producing Yeast

In a final aspect, the use of a disrupted, partially deleted or completely deleted CIT1 and/or ACH1 yeast allele is provided to develop an acetic acid producing yeast, more particularly an S. boulardii yeast. This is equivalent as saying that a method of producing an acetic acid producing yeast (more particularly an S. boulardii yeast) or of producing an engineered S. boulardii yeast with improved probiotic activity is provided. The method comprises the step of disrupting, partially deleting or completely deleting Cit1 and/or Ach1 function in yeast, more particularly an S. boulardii yeast. In one embodiment, this can be achieved by mutating or deleting all CIT1 and/or ACH1 alleles in said yeast as explained earlier in this document. In another embodiment, said method further comprises the step of enhancing the expression of ALD4 in said yeast, more particularly S. boulardii yeast. This is equivalent as saying that the combined use of a disrupted, partially deleted or completely deleted CIT1 and/or ACH1 yeast allele with enhanced expression of ALD4 is provided to develop an acetic acid producing yeast, more particularly an S. boulardii yeast. Enhanced expression of ALD4 can be achieved as explained earlier, for example by expressing at least one chimeric gene construct comprising an ALD4 allele.

The application also provides an acetic acid producing yeast, more particularly an S. boulardii yeast obtained by the above described method.

Also, the use is provided of a yeast strain having a statistically significant reduced expression of CIT1 and/or ACH1 for the production of acetic acid. In one embodiment, the yeast further has a statistically significant enhanced expression of ALD4. Reducing expression of CIT1 and/or ACH1 can for example be achieved by disrupting, partially deleting or completely deleting all CIT1 and/or ACH1 yeast alleles as explained herein. Enhanced expression of ALD4 can be achieved as explained earlier, for example by expressing at least one chimeric gene construct comprising an ALD4 allele. In particular embodiments, said yeast comprises at least one WHI2 wild-type allele. In another particular embodiment, said yeast further comprises a homozygous or hemizygous SDH1 mutant allele, more particularly a loss-of-function SDH1 allele, even more particularly the loss-of-function SDH1 allele as depicted in SEQ ID No. 13. In another particular embodiment, said yeast is useful for acetic acid production, including, but not limited to Saccharomyces, Zygosaccharomyces, Brettanomyces and Kluyveromyces. Preferably, said yeast is a Saccharomyces sp., even more preferably it is a Saccharomyces cerevisiae sp., most preferably it is a S. boulardii.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### Examples

### Example 1: Enhancing the Acetic Acid Production of the Sb.P Strain

Sb.P, a natural S. boulardii isolate, is homozygous for the recessive mutation whi2^{s270*}, which makes it able to accumulate unusually high amounts of acetic acid compared to other S. boulardii strains (Offei et al 2019). The causative allele whi2^{s270*}, harbors a temperature sensitive mutation resulting in growth deficiency on acetic acid at 37° C. However, this mutation also leads to acetic acid sensitivity. As a result, Sb.P exhibited only 20% cell viability at the end of a fermentation at 37° C., while the other tested S. boulardii strains, including ENT, which are heterozygous for WHI2/whi2* as well as the S. cerevisiae strain ER (WHI2/WHI2), all exhibited close to 100% cell viability (Offei et al 2019).

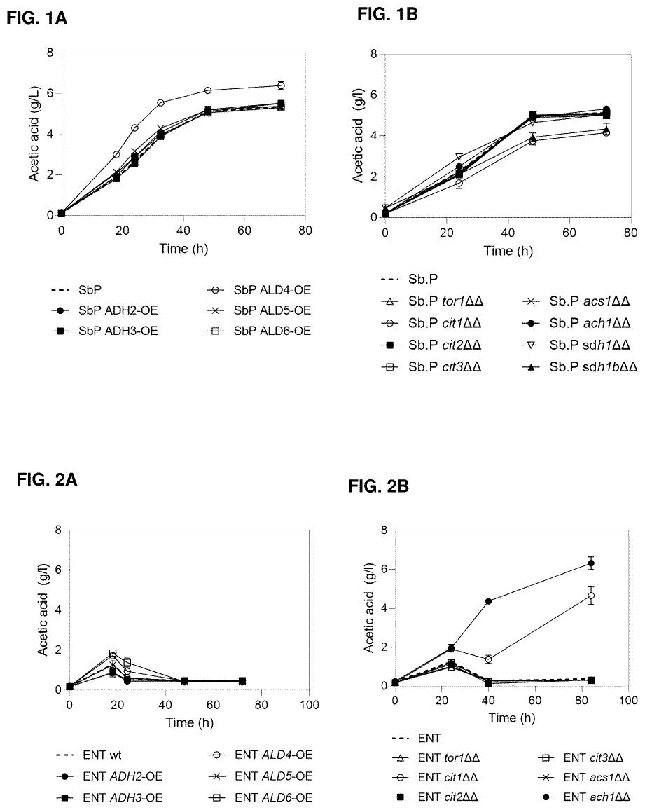

In order to increase acetic acid production by Sb.P, we have tested 13 candidate genes, being 5 targets for overexpression (ADH2, ADH3, ALD4, ALD5, ALD6) and 8 targets for deletion (CIT1, CIT2, CIT3, ACH1, SDH1, SDH1b, ACS1 and TOR1). Among all tested deletions and overexpressions, the only modification able to increase acetic acid accumulation in Sb.P was the overexpression of the ALD4 gene (FIG. 1A). SDH1b and CIT1 deletion resulted in decreased acetic acid accumulation, the latter also negatively affected propagation. All the other modifications did not affect acetic acid accumulation or cell growth (FIG. 1B). However, due to the acid sensitivity of Sb.P, we may not be able to further increase its acetic acid production without losing viability.

### Example 2. Impairing the TCA Activity Shifts Acetate Production in the ENT Strain from Transient to Permanent

With the aim of constructing a very high acetate producing strain without the acid sensitivity displayed by Sb.P, we have tested the same genetic modifications in the ENT background. Among the overexpression targets, ALD4 and ALD6 could only slightly improve acetate accumulation (FIG. 2A). In addition, the accumulation remained transient. Interestingly, when the CIT1 or ACH1 gene were deleted, a significant and unexpected effect was observed: the yeast cells switched from transient to permanent acetate accumulation in YPD2% (FIG. 2B). This in strong contrast to CIT1 deletion in the Sb.P strain (FIG. 1B).

CIT1 encodes a mitochondrial citrate synthase that catalyzes the first step of the TCA cycle, the condensation of acetyl coenzyme A and oxaloacetate to form citrate. CIT1 deletion in ENT resulted in a final acetate accumulation (at 72 h) of about the same level as that of wild-type Sb.P. However, this accumulation happened at much slower rate (FIG. 2B). As observed in Sb.P, CIT1 deletion in the ENT background also resulted in a partial growth defect. During propagation in YPD2% at 30° C., the ENT cit1ΔΔ strain was only able to reach about half of the cell density of ENT wild type in stationary phase.

On the other hand, ACH1 deletion did not interfere with propagation capacity. The ENT strain in which ACH1 was knocked-out (named ENT1 from hereon) accumulated higher levels of acetate compared to Sb.P. Interestingly, ACH1 deletion in Sb.P did not have any effect on acetate production and all transformants showed a similar acetate accumulation pattern as displayed by the wild type Sb.P strain (data not shown).

### Example 3. ALD4 Overexpression Further Enhances Acetate Accumulation

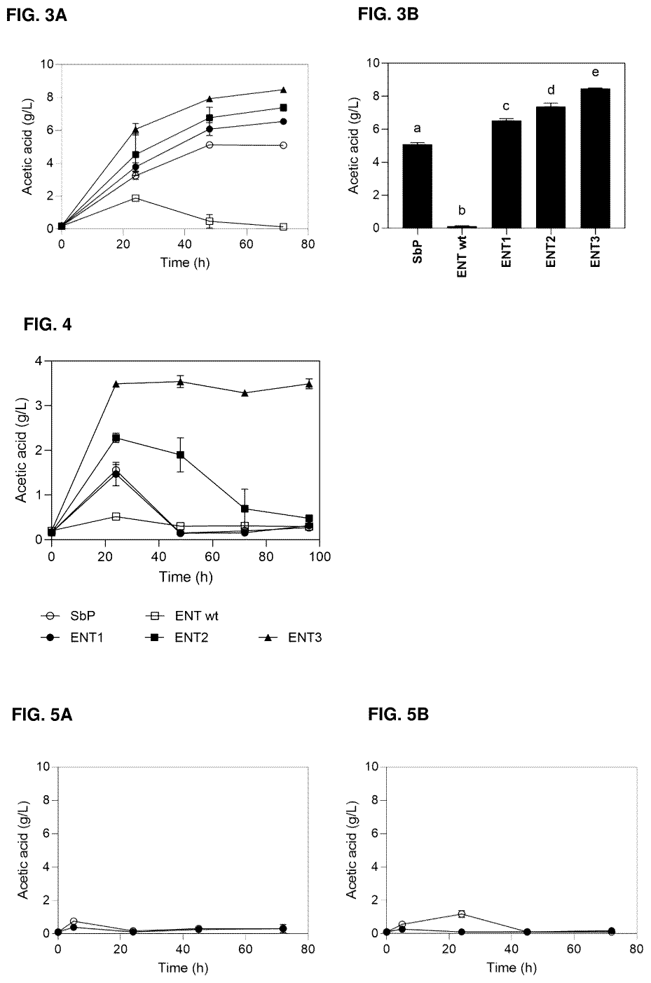

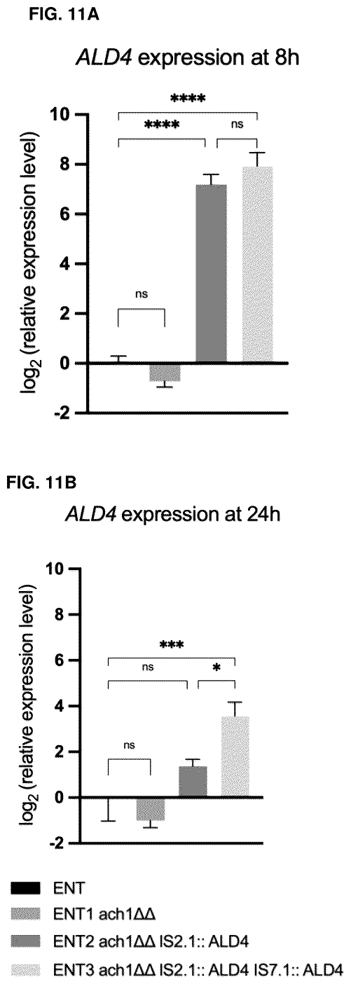

The overexpression of the ALD4 gene was the only modification able to further increase acetate production in Sb.P (FIG. 1A). ALD4 is a mitochondrial aldehyde dehydrogenase required for growth on ethanol. It catalyzes the conversion of acetaldehyde to acetate and its expression is repressed by glucose. However, when overexpressed in the ENT strain under the strong and constitutive TEF1 promoter, ALD4 only gave a slightly higher acetate accumulation. Moreover the accumulation was transient (FIG. 2A). Next, we expressed the ALD4 gene in the ENT1 strain (i.e. ENT ach1ΔΔ). When two copies of the ALD4-OE construct were integrated in the genome (resulting in strain ENT2), the strain produced 14% more acetate compared to ENT1. Upon integration of two more copies (resulting in strain ENT3) the strain produced 30% more acetate than ENT1 and 70% more compared to the Sb.P strain (FIGS. 3A and B). It was confirmed that the integration of more ALD4 copies significantly increased the ALD4 expression in the yeast strains at both exponential and early stationary phase. (FIG. 11).

In Sb.P, ALD4-OE was enough to improve acetate accumulation (FIG. 1A). In the ENT strain however, ALD4-OE has to be combined with impaired TCA cycle activity to induce a strong increase in acetate accumulation. Interestingly, the obtained acetate production became permanent in contrast to the transient acetate production in ENT wild-type.

### Example 4. ENT3 Permanently Accumulates Acetate when Growing in Low Glucose Concentrations

Luminal glucose concentrations were reported to range from 0.2 to 48 mM (0.036%—0.86%) under all physiological conditions and stated not to exceed 100 mM (1.8%) even under the most unphysiological condition examined (Ferraris et al 1990 Am J Physiol 259). However, Ferraris et al. measured the overall luminal glucose concentration by simply emptying the gut content, while the glucose concentration in the unstirred layer of the brush border may be much higher because all major carbohydrases are attached to the epithelial cell membrane (Kellett 2001 J Physiol 531). Direct measurements are difficult since glucose from hydrolysis is present only transiently due to its constant uptake and diffusion. Nevertheless, indirect measurements from the rate of membrane hydrolysis of maltose suggest that the local concentration could reach levels of the order of 300 mM (5.4%) (Pappenheimer 1998 Comp Biochem Physiol A Mol Integr Physiol 121).

We have previously reported that higher sugar levels (4%) lead to very high acetic acid accumulation in all S. boulardii strains but not in most S. cerevisiae strains tested (Offei et al. 2019). It means that after a meal, when the glucose concentration is high, all S. boulardii strains are capable of accumulating acetic acid. Since sugar concentrations can also be low in the brush border for extended times between meals, we have now also investigated acetate accumulation by the different S. boulardii strains in low glucose concentrations.

When growing on YPD 0.9% (50 mM glucose), we observed that Sb.P can only transiently accumulate acetate while ENT wild type does not accumulate significant levels. When the ACH1 gene was deleted (resulting in strain ENT1), the ENT1 strain showed the same profile as Sb.P, with a maximal acetate production of about 1.5 g/l at 24 h. Considerably higher but still transient accumulation was observed in ENT2. Surprisingly, the ENT3 strain was the only strain tested capable of permanently accumulating about 3.5 g/l of acetate (FIG. 4).

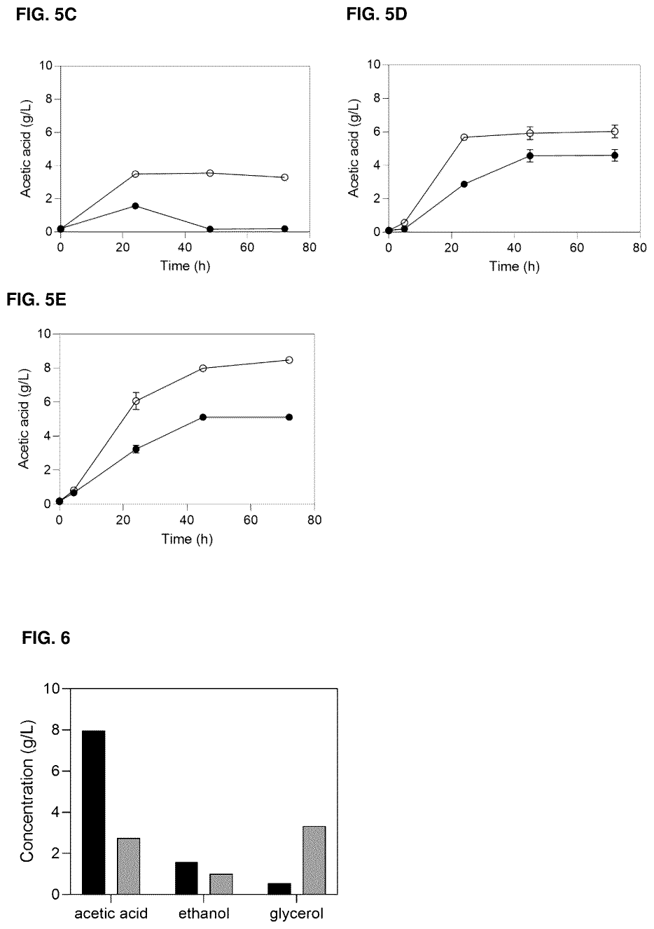

Next, we compared Sb.P and ENT3 over a range of glucose concentrations and we observed that ENT3 is consistently superior to Sb.P at all glucose concentrations tested. At the lowest tested glucose concentration (5 mM), none of the strains was able to accumulate acetate (FIG. 5A). At 20 mM (0.36%), only ENT3 was able to accumulate acetate, although still transiently (FIG. 5B) and at 50 mM (0.9%) ENT3 could permanently accumulate acetate while Sb.P showed lower and still transient accumulation (FIG. 5C). From 75 mM (1.35%) both strains could accumulate acetate permanently but ENT3 consistently showed superior accumulation (FIG. 5D) and the difference between the two strains got even more pronounced at 110 mM of sugar (2%, FIG. 5E).

We hypothesize that ENT3 may be able to exert a stronger and longer-lasting probiotic effect as it is able to produce the bacteria-inhibiting compound acetic acid even when glucose availability is low, which is the case during the fasted state in between meals.

In order to assess if the high acetate phenotype of ENT3 was transferable, we inserted the same modifications in other wild type S. boulardii strains (WHI2/whi2*), namely UL and 7103. As shown in FIG. 13, a very high acetic acid accumulation was achieved in both engineered UL and 7103 strains, indicating a generic effect of the Ach1 deletion and ALD4 overexpression.

### Example 5. ADH1 Deletion Resulted in Decreased Acetate Accumulation in Favor of Higher Glycerol Production

ADH1 encodes an alcohol dehydrogenase that reduces acetaldehyde to ethanol. The opposite reaction is catalyzed by the Adh2 enzyme that oxidizes ethanol to acetaldehyde, which is then converted into acetate. By deleting ADH1 in ENT3, we expected to force the glycolytic flux towards acetate. Instead, the ENT3 adh1ΔΔ strain produced less acetate than ENT3 and the carbon was deviated to a large extent into higher glycerol production (FIG. 6).

### Example 6. ENT3 Exhibits Inhibitory Capacity Against Gut-Isolated Pathobiont Bacteria

We have previously shown that acetic acid at the concentration of 6 g/l, as well as Sb.P fermentation medium supernatant, could inhibit the growth of Escherichia coli MG1655 (Offei et al 2019). Here we have investigated the acetic acid efficiency for antimicrobial activity against potential pathobionts isolated from the gut. We have tested different concentrations of acetic acid as well as Sb.P and ENT3 fermentation medium supernatants by the agar-well diffusion assay in the presence of gut bacteria. For this test, appropriate agar medium (Schaedler) was inoculated with each one of the tested bacterial strains. Wells were punched into the agar and filled with: 100 μg/ml ampicillin (control), acetic acid at 3, 6, 9 or 12 g/l in YP pH 4 or fermentation medium supernatant of the Sb.P and ENT3 strains. Two sets of plates were prepared, one of which was incubated inside an anaerobic jar. Plates were incubated at 37° C.

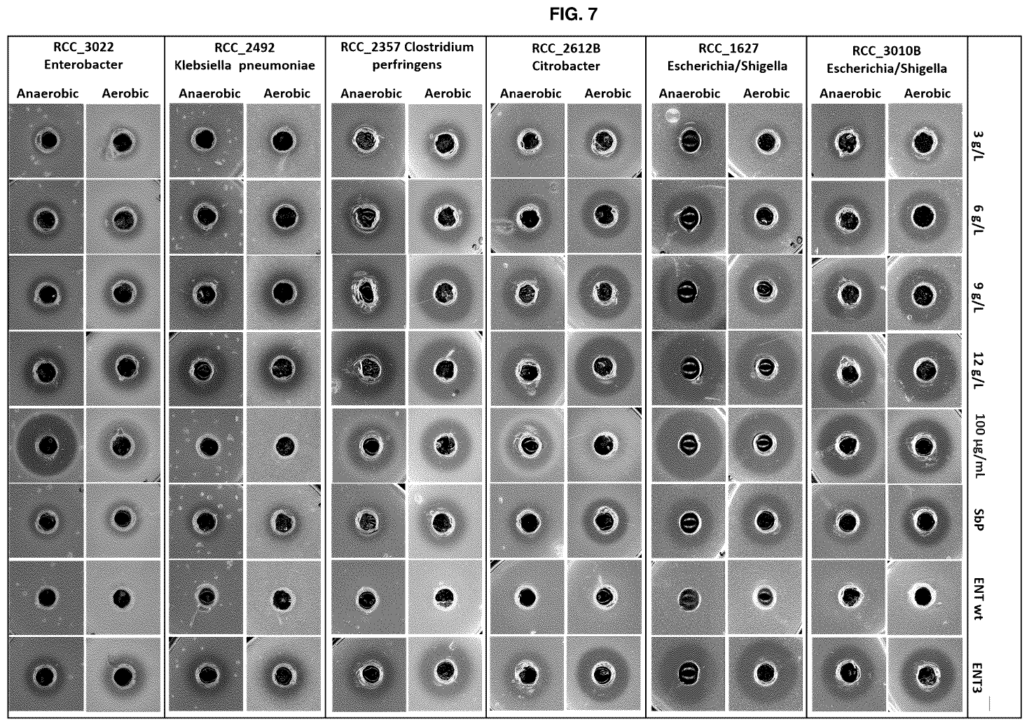

We observed that acetic acid was effective against all tested potential pathobionts (FIG. 7). As expected, the higher the acetic acid concentration, the larger was the inhibition zone, showing that the antimicrobial activity was due to the acetic acid and not to the YP pH4 medium. All tested strains except Klebsiella pneumoniae were inhibited by ampicillin and the sensitivity of the strains was higher in the absence of oxygen, as shown by the larger inhibition zone (FIG. 7). Acetic acid efficacy, however, was unaffected by oxygen. This is especially interesting knowing that in dysbiosis the epithelial barrier is compromised causing higher release of oxygen into the gut.

The Sb.P and ENT3 supernatants were also effective against all tested strains. However, ENT3 exhibited in all cases stronger antimicrobial potency compared to Sb.P. ENT3 supernatant behaved similarly to the highest administered acetic acid concentration and inhibited the ampicillin resistant Klebsiella pneumoniae as well (FIG. 7). The inhibition by ENT3 supernatant was neither affected by the presence of oxygen. A clear decrease in supernatant pH from ENT3 cultures can be observed when compared to that of ENT or SbP cultures (Table 1).

TABLE 1
Supernatant pH and acetic acid (AA) concentrations
after propagation in YPD2%, 37° C., 72 h.

| Strain | AA (g/L) at 72 h in YPD2% | Supernatant pH |
|---|---|---|
| SbP | 5.0 | 4.07 |
| SbP SDH1^{sc} | 0.25 | 4.89 |
| ENT | 0.20 | 4.69 |
| ENT3 | 8.50 | 3.73 |

### Example 7. ENT was Engineered for High Acetic Acid Production without Compromising Cell Viability at Low pH

An ideal probiotic micro-organism should be tolerant to the low pH of gastric fluid in order to survive gastric passage as some probiotic actions depend on cell viability or metabolic activity of the yeast, such as secretion of anti-toxin proteins and anti-microbial agents like acetic acid. It has been demonstrated that an S. boulardii CNCM 1-745 strain (same strain as ENT) exhibits superior tolerance (75% viability) to low pH when compared to the laboratory S. cerevisiae strain W303 (30% viability) in simulated gastric juice (pH 2) (Fietto et al 2004 Can J Microbiol).

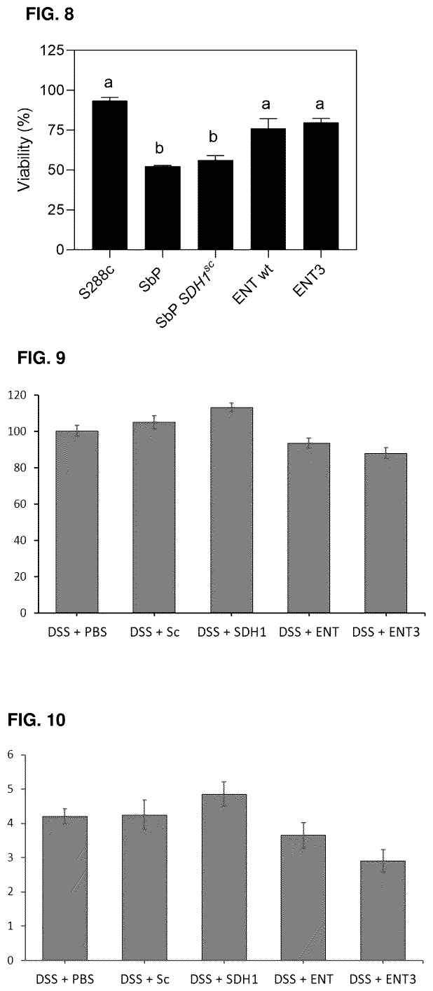

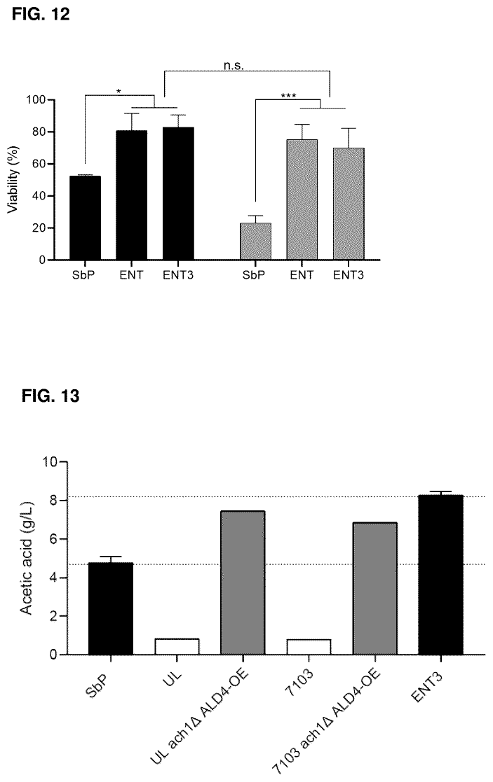

In order to investigate whether the engineering of ENT for high acetic acid accumulation had compromised its tolerance to low pH, we have simulated gastric passage by exposing strains to saline solution (NaCl 0.5%) at pH 1.7 for 3 h at 37° C. This pH was chosen as this value was reported to be the median gastric pH in humans in the fasted state, with interquartile range from 1.4 to 2.1 (Dressman et al 1990 Pharm Res 7). For comparison, we have included an S. cerevisiae strain (S288c), as well as Sb.P and the Sb.P SDH1S strain in which acetic acid production is abolished. Viability was determined by colony counting on nutrient agar plates of cells that had been exposed for 3 h to acidic saline (NaCl 0.5%, pH1.7) compared to 3 h exposure to saline at pH 6.8. In this way, only the effect of low pH is taken into consideration. While a statistically significant drop in viability of about 50% was observed for Sb.P and Sb.P SDH1S compared to S288c, the engineering of the ENT strain for high acetate production did not significantly compromise its resistance to low pH. Both ENT wild type and ENT3 exhibiting about 80% viability (FIG. 8). Difference in viability between SbP and ENT3 becomes even more pronounced at pH 1.2. While ENT and ENT3 do not show significant difference in viability compared to pH 1.8 (approx. 73%), SbP viability was about 20% (FIG. 12). The pH sensitivity displayed by Sb.P appears to be due mainly to the whi2^{S270*} allele. When this allele is replaced by the S. cerevisiae wild type allele WHI2, the tolerance to low pH is restored. The whi2^{S270*} allele encodes a truncated protein and caused a similar phenotype as whi24 (Offei et al 2019). Chen and collaborators (2018, PLoS Genet 14) also observed that the whi24 strain had substantially higher sensitivity to acetic acid than the wild type. As ENT and its engineered strain ENT3 are heterozygous for WHI2/whi2*, they do not show the high acid sensitivity displayed by the Sb.P strain, which harbors two copies of the mutated allele whi2*.

### Example 8. ENT3 Attenuates Inflammation in DSS-Induced Colitis

To evaluate the potency of the engineered ENT strains in vivo, the effect of S. boulardii (Sb) strains with different levels of acetate production was assessed in a DSS-induced mouse model of colitis. Nine week old female C57/B16 mice (N=120; 10 mice per group) were allocated to treatment groups receiving either regular drinking water or DSS in combination with (1) PBS, (2) Baker's yeast (non-probiotic control), (3) SDH1, a non-acetate producing Sb strain, (4) ENT, the Sb Enterol strain (probiotic control) and (5) ENT3, a super high acetate producing Sb strain (8.5 g/l). Disease activity including weight loss, diarrhoea and the presence of occult blood was scored daily. On day 7, the DSS groups were transferred to regular drinking water and on day 14 mice were sacrificed to evaluate macroscopical damage of the gut. Statistics were performed using GraphPad Prism 9 (One-way ANOVA for parametric data and Kruskal-Wallis tests for non-parametric data).

Disease activity, determined by the area under the curve, in the mice treated with the ENT3 strain was significantly lower compared to that from mice treated with the negative controls PBS or Baker's yeast (p=0.015 and p=0.011 respectively) and compared to the Sb strain SDH1 (p=0.0001). The disease activity in mice treated with ENT3 was also lower compared to that from mice treated with the non-engineered ENT strain (FIG. 9).

At sacrifice, gut macroscopical damage scores were significantly lower for ENT3 treatments compared to Sb SDH1 and the negative controls. The macroscopical damage score for ENT3 was also lower compared to that of ENT treatment (FIG. 10).

### Materials and Methods

Strains, media and culture conditions: The strains used in this application are listed in Table 1. Yeast cells were propagated in YPD medium containing 10 g/L yeast extract, 20 g/L bacteriological peptone, and 20 g L glucose at 30C or 35C. To make solid nutrient plates the media were supplemented with 1.5 g/L bacto agar. Where appropriate, the medium was supplemented with antibiotics: for S. cerevisiae i.e. 200 mg/L geneticin, 300 mg/L hygromycin B or 100 mg/L nourseothricin, for S. boulardii i.e. 50 mg/L geneticin, 75 mg/L hygromycin B, 2 mg/L nourseothricin. The gut isolated bacterial strains were pre-grown in Schaedler broth (26.5 g/L, Thermo Scientific).

List of yeast strains

| Strain | Genotype | Description | Origin |
|---|---|---|---|
| S. boulardii | Wild type | Natural isolate accumulating | Lene Jespersen, |
| Sb. P | | unusually high levels of acetic | University of |
| | | acid in YPD2%. sensitive to | Copenhagen, Denmark |
| | | low pH. | |
| Sb. P | SDH1^{Y202H, Y317F}/SDH1^{Y202H, Y317F} | Sb. P harboring two copies of | Offei et al. 2019 |
| SDH1^{Sc} | | the SDH1 allele of S288c. | |
| | | Does not accumulate acetic | |
| | | acid. | |
| S. boulardii | Wild type | Commercial S. boulardii. | Offei et al. 2019 |
| ENT | | Shows transient acetic acid | |
| | | production in YPD2%. | |
| ENT1 | ACH1ΔΔ | High acetic acid in YPD2%, | This work |
| | | does not accumulate acetic | |
| | | acid in YPD0.8% | |
| ENT2 | ACH1ΔΔ | High acetic acid in YPD2%. | This work |
| | TEFp-ALD4-CYC1t::IS2.1 | Transient acetic acid | |
| | | accumulation in YPD0.8% | |
| ENT3 | ACH1ΔΔ | Very high AA in YPD2%. | This work |
| | TEFp-ALD4-CYC1t::IS2.1 | Permanent acetic acid | |
| | TEFp-ALD4-CYC1t::IS7.1 | accumulation in YPD0.8% | |

| Strain | Source | Reference | Origin |
|---|---|---|---|
| UL | University of | Offei etal., 2019 | Unknown. Source of reference: P. |
| | Manchester, UK | Edwards-Ingram et al. 2007 | Niederberger, Nestle (Edwards- |
| | | | Ingram et al. 200) |
| 7103 | University of | Offei etal., 2019 | Ultra-Levure batch 7103, |
| | Copenhagen, Denmark | van der Aa Kühle et al., 2005 | Laboratoires Biocodex |

General molecular biology methods: Yeast genomic DNA was extracted with Phenol/Chloroform/Isoamyl alcohol (25:24:1) and, where required, further purified by ethanol precipitation. PCR was performed according to manufacturer's specifications with Standard Taq DNA polymerase for diagnostic purposes or Q5 high-fidelity DNA polymerase for sequencing or amplification of donor DNA (New England Biolabs). Either the LiOAC/SS-DNA/PEG protocol or electroporation were used as transformation methods (Bentuil et al 2010 Protein Eng Des Sel 23; Demeke et al 2013 Biotechnol Biofuels 6).

Acetic acid accumulation assay: Overnight yeast pre-cultures were adjusted to an OD₆₀₀ of 0.5 (corresponding to 4×10⁶ CFU/mL) in 50 ml YPD in a 300 ml Erlenmeyer flask. Flasks were incubated by shaking at 200 rpm and 37° C. in a shaking incubator for 48 or 72 h. To obtain cell-free culture supernatants, aliquots of yeast cultures were withdrawn from the flasks and centrifuged at maximum speed (14,000 rpm) for 5 min. The supernatants were used for agar-well diffusion assays or subjected to High Performance Liquid Chromatography (HPLC) to determine the acetic acid concentration. For time-course measurements, samples were withdrawn from the cultures every 12 h for further analysis.

Agar-well diffusion assays: Round petri dishes containing 20 mL Schaedler agar (15 g bacto agar/L Schaedler broth) were overlayed with molten soft Schaedler agar (7.5 g bacto agar/L Schaedler broth) inoculated with each one of the tested bacterial strains at a concentration of approximately 5.10⁴ cells/mL. Wells were punched into both agar layers. The resulting agar discs were carefully removed from each well with a pair of sterile thongs and discarded. Each well was then filled with 200 μl of testing solution. The testing solutions were: ampicillin (100 μg/mL), pure acetic acid solutions (at 3, 6, 9 or 12 g/L in YP pH 4) and cell-free supernatant (from Sb.P, ENT and ENT3 cultures). Two sets of plates were prepared, one of which was incubated inside an anaerobic jar. Plates were incubated at 37° C. for 24-48 h.

Yeast tolerance to low pH: Overnight cultures were harvested, adjusted to OD_{600 nm}=1 by centrifugation at 3000 rpm for 5 min, washed with distilled water once, and incubated at 37° C. for 3 h in (i) a simulated gastric environment constituted by an aqueous solution containing 5 g/L NaCl, pH 1.7 (ii) a control saline containing 5 g/L NaCl, without pH adjustment (pH 6.8). Viability was determined by colony counting on nutrient agar plates of cells that had been exposed for 3 h to acidic saline (NaCl 0.5%, pH1.7) compared to 3 h exposure to saline at pH 6.8. In this way, only the effect of low pH is taken into consideration and any possible osmotic effect is excluded.

Genotyping by allele-specific PCR: Allele-specific PCR for each block of deleted genes or each individual gene in the RHA assay and allele replacement was performed by pairing a forward primer, containing either the SBERH6 or S288c nucleotide as the 3′ terminal nucleotide, with a common reverse primer. To increase specificity, for some primers, an additional single nucleotide artificial mismatch was added within the three bases closest to the 3′ end. The annealing temperature for each set of primers was optimized by gradient PCR using genomic DNA of both parents, so as to allow only hybridization with primers containing the exact complement.

CRISPR/Cas9 mediated gene exchange: The gRNA plasmid and the Cas9 expression plasmids used in this study were based on the paper by DiCarlo et al. (2013 Nucleic Acids Research 41), and were recently described by Holt and coworkers. Allele replacement of SDH1 and WHI2 was performed in a stepwise manner. First, a dominant selection marker (ble^{r} or NatMX4), or both when a diploid strain was modified, flanked by gRNA recognition sites, G1, was used to delete the region of interest. The selection markers were amplified from plasmid pTOPO_G1-NatMX4-G1 or pTOPO-G1-Ble^{R}-G1, with primers containing tails, homologous to the regions flanking the targeted region. Correct integration of the cassettes was confirmed by PCR. For each replacement, to obtain independent replacement strains, three successful transformants were selected and all following steps were performed in parallel. Next, pTEF-Cas9-KanMX4, the plasmid harboring Cas9, was introduced. Finally, the plasmid expressing the gRNA, pgRNA-G1-HphMX, that specifically targets the G1 sequence (5′-GGCTGATTTTCGCAGTTCGGGGG-3′) flanking the marker, was introduced together with donor DNA to repair the double stranded break by homology directed repair. The design of this gRNA was based on the finding of Farboud and Meyer that Cas9-mediated DNA cleavage was enhanced at this G1 site due to the presence of a GG dinucleotide at the 3′ end of the protospacer. This gRNA was checked for potential off targeting with BLAST. For SDH1, the region from 99 bp upstream of the first non-synonymous SNP (c.604G>A), to 91 bp downstream of the second non-synonymous mutation (c.950A>T), was replaced. While for WHI2, the entire region between the gene downstream and upstream of WHI2 was replaced. Repair templates were amplified from genomic DNA of SBERH6 or S288c and variant whi2^{Stop287S} was amplified with genomic DNA of S. boulardii strain LSB as template. For SDH1, repair templates harboring just one of the two non-synonymous mutations were constructed. For this purpose the left and right part of the repair template for SDH1 were amplified separately with genomic DNA of SBERH6 or S288c as template. Next, these fragments were joined into a single repair template by fusion PCR using an overlapping sequence between the two fragment, yielding two repair templates for SDH1 that each contained one of the two non-synonymous SNPs. Also, for each strain that was modified, re-integrants, where the native DNA was used as a repair template, were constructed. Replacement of the NatMX4 or ble^{R} marker resulted in sensitivity to nourseothricin or phleomycin respectively and was assessed by spot assay. The presence of the introduced variant was verified by PCR and next, by sub-culturing three times in YPD, the plasmids were lost. Plasmid loss was verified by spot assay on YPD supplemented with hygromycin B or geneticin. Finally, the sequences of the replaced region and its surroundings were confirmed by Sanger sequencing.

### Gene Deletion Experiments

Two gRNAs were used targeting each gene. The first gRNA targets within the first nucleotides in the open reading frame (ORF) and the second gRNA targets within the last ones. An 80-bp oligomer is given as repair template, consisting of 40 bp immediately upstream and 40 bp immediately downstream the ORF. As a result, the ORF is removed and no exogenous DNA is inserted.

### Overexpression Experiments

Each overexpression target gene has its original sequence amplified from Sb.P and cloned under the strong and constitutively expressed TEF1 promoter and the CYC1 terminator. Each gene was tested in Sb.P and ENT strains. All tested genes were individually inserted in the same previously tested neutral site, named IS2.1. For the ENT3 construction, ALD4-OE copies were inserted at the sites IS2.1 and 157.1.

### SEQUENCES

SEQ ID No. 1: DNA sequence CIT1 from Saccharomyces boulardii
SEQ ID No. 2: protein sequence Cit1 from Saccharomyces boulardii
SEQ ID No. 3: DNA sequence ACH1 from Saccharomyces boulardii
SEQ ID No. 4: protein sequence Ach1 from Saccharomyces boulardii
SEQ ID No. 5: DNA sequence ALD4 from Saccharomyces boulardii
SEQ ID No. 6: Protein sequence Ald4 from Saccharomyces boulardii
SEQ ID No. 7: DNA sequence ALD6 from Saccharomyces boulardii
SEQ ID No. 8: Protein sequence Ald6 from Saccharomyces boulardii
SEQ ID No. 9: DNA sequence WHI2 from Saccharomyces boulardii
SEQ ID No. 10: Protein sequence Whi2 from Saccharomyces boulardii
SEQ ID No. 11: DNA sequence SDH1 allele from Saccharomyces cerevisiae
SEQ ID No. 12: Protein sequence Sdh1 from Saccharomyces cerevisiae
SEQ ID No. 13: DNA sequence of the SDH1^{T950A} allele from Saccharomyces boulardii
SEQ ID No. 14: Protein sequence of Sdh1^{F317Y} from Saccharomyces boulardii

1. A mutant Saccharomyces boulardii yeast comprising a compromised, partially abolished or completely abolished Acetyl-CoA hydrolase 1 (Ach1) or Citrate synthase 1 (Cit1) function as compared to wild-type Saccharomyces boulardii yeast.

2. The mutant S. boulardii yeast of claim 1, wherein the mutant S. boulardii comprises a homozygous loss-of-function ACH1 and/or CIT1 mutant allele.

3. The mutant S. boulardii yeast of claim 1, in which all ACH1 and/or CIT1 alleles are deleted.

4. The mutant S. boulardii yeast of claim 1, wherein ALDEHYDE DEHYDROGENASE 4 (ALD4) expression is statistically significantly enhanced compared to an isogenic control S. boulardii yeast.

5. The mutant S. boulardii yeast of claim 1, wherein the mutant S. boulardii comprises at least one chimeric gene construct comprising at least one ALD4 allele for ALD4 overexpression.

6. (canceled)

7. (canceled)

8. The mutant S. boulardii yeast of claim 5, wherein the chimeric gene construct comprises the TEF1 promoter driving the expression of the ALD4 allele.

9. A mutant Saccharomyces boulardii yeast producing at least 0.5 g/l acetic acid at 37° C. in low glucose medium of about 20 mM glucose.

10. The mutant Saccharomyces boulardii yeast of claim 9, wherein the mutant S. boulardii produces at least 1 g/l acetic acid at 37° C. in low glucose medium of about 50 mM glucose.

11. The mutant S. boulardii yeast of claim 9, wherein ALD4 expression during exponential growth phase is at least 2-fold higher compared to a control S. boulardii yeast.

12. The mutant S. boulardii yeast of claim 9, wherein the mutant S. boulardii yeast is genetically engineered to have increased expression of ALD4 compared to a non-engineered S. boulardii control yeast.

13. The mutant S. boulardii yeast according of claim 9, wherein the mutant S. boulardii comprises at least two chimeric gene constructs comprising at least one ALD4 allele.

14. (canceled)

15. The mutant S. boulardii yeast of claim 13, wherein the chimeric gene constructs comprise the TEF1 promoter driving the ALD4 expression.

16. The mutant S. boulardii yeast of claim 1, wherein the mutant S. boulardii yeast is not Sb.P strain.

17. The mutant S. boulardii yeast of claim 1 further comprising at least one WHISKEY2 (WHI2) wild-type allele.

18. The mutant S. boulardii yeast of claim 1, further comprising a homozygous or hemizygous SUCCINATE DEHYDROGENASE1 (SDH1) mutant allele.

19. The mutant S. boulardii yeast of claim 18, wherein the SDH1 mutant allele is a loss-of-function allele.

20. The mutant S. boulardii yeast of claim 18, wherein the SDH1 mutant allele comprises SEQ ID No. 13.

21. The mutant S. boulardii yeast of claim 1, said yeast being the CNCM I-475, UL, 7103 or the ENT strain.

22. A dietary supplement or pharmaceutical composition comprising the mutant S. boulardii yeast of claim 1.

23. (canceled)

24. (canceled)

25. (canceled)

26. (canceled)

27. (canceled)


SEQUENCE LISTING (15 sequences)

SEQ ID NO: 1 - DNA, 1440 bp, Saccharomyces boulardii
  source 1..1440: mol_type = genomic DNA
atgtcagcgatattatcaacaactagcaaaagtttcttatcaaggggctccacaagacaatgtcaaaatatgcaaaaggctctttttgcactattgaatgctcgccactatagtagcgcctccgaacaaacgttgaaggagagatttgctgaaattatcccagcaaaggcagaagaaattaaaaaattcaagaaagaacacggtaaaaccgttattggtgaagttcttttggagcaagcttatggtggtatgagaggtattaaaggccttgtttgggaaggttccgtgttagaccccgaagaaggtattagatttaggggtcgtactattccagaaattcaaagggaactaccaaaggctgagggtagtacagaacctttgccagaagctttattttggttgcttttgactggtgaaatacctactgacgctcaagttaaagccctttctgctgatttagctgccagatcagaaattccagagcacgttatccaacttttagatagcctcccaaaagatctacatccaatggcgcaattttctattgccgtgactgctttagaaagcgagtctaagtttgccaaagcatatgctcaaggtgtatccaagaaagaatattggagctatacatttgaagattcgttagatctgctgggtaaattacctgttattgcttccaaaatttatcgtaatgtgttcaaggatggtaaaattacttcaaccgatcctaatgctgactatggtaaaaatttggcccaacttttgggctacgaaaacaaggattttattgacttaatgagactatatttaactattcattctgatcatgaaggtggtaacgtttctgcccatactacacatttagtgggttctgccttatcttcgccatacttatctttggccgctggtttgaatggtttagctggcccattacatggtcgtgccaatcaagaagttttagaatggctatttaaattgagagaagaagtgaaaggtgactattcaaaagaaacaattgaaaagtacttgtgggatactttgaacgcagggagagttgttcctggttatggccatgcggttttgagaaaaactgatcctcgttatacggctcaacgtgaattcgcattgaaacatttcccagattacgagttatttaagttggtctccaccatttatgaagttgccccaggggttttaactaagcatggtaaaactaagaacccatggccaaatgttgattcacattccggtgttttattgcaatactatggtctaactgaggcttcgttctacactgtattgtttggtgttgccagagctattggtgtgttaccccaattaatcatcgatagggctgttggtgctccaatcgaaaggccaaaatcattctccaccgaaaaatacaaggagttggtaaagaaaatcgaaagtaagaactaa

SEQ ID NO: 2 - protein, 479 aa, Saccharomyces boulardii
MSAILSTTSKSFLSRGSTRQCQNMQKALFALLNARHYSSASEQTLKERFAEIIPAKAEEIKKFKKEHGKTVIGEVLLEQAYGGMRGIKGLVWEGSVLDPEEGIRFRGRTIPEIQRELPKAEGSTEPLPEALFWLLLTGEIPTDAQVKALSADLAARSEIPEHVIQLLDSLPKDLHPMAQFSIAVTALESESKFAKAYAQGVSKKEYWSYTFEDSLDLLGKLPVIASKIYRNVFKDGKITSTDPNADYGKNLAQLLGYENKDFIDLMRLYLTIHSDHEGGNVSAHTTHLVGSALSSPYLSLAAGLNGLAGPLHGRANQEVLEWLFKLREEVKGDYSKETIEKYLWDTLNAGRVVPGYGHAVLRKTDPRYTAQREFALKHFPDYELFKLVSTIYEVAPGVLTKHGKTKNPWPNVDSHSGVLLQYYGLTEASFYTVLFGVARAIGVLPQLIIDRAVGAPIERPKSFSTEKYKELVKKIESKN

SEQ ID NO: 3 - DNA, 1581 bp, Saccharomyces boulardii
  source 1..1581: mol_type = genomic DNA
atgacaatttctaatttgttaaagcagagagttaggtatgctccctatctgaaaaaagttaaggaagctcacgagcttattccattgttcaagaatggtcagtatcttgggtggtccggttttacaggagtgggtactcccaaggcagtgccggaggcactgatagatcacgtggagaagaacaatttacaagggaagttgagattcaacctttttgttggagcttctgctggtccagaggaaaaccgttgggctgaacacgacatgatcattaagagagcccctcatcaagtagggaaacccattgcaaaggcaattaaccagggtagaattgagttctttgataaacatctgtccatgttccctcaggatctgacatacgggttctacaccagggaaagaaaagacaacaaaatccttgattatactataatcgaggcaacggccattaaagaggacgggtctatcgtcccaggtccctctgtcggtggttctccagaattcattacagtcagtgataaagtgattattgaggttaacacggctacgccttcgttcgagggtattcacgatatagacatgcccgtgaacccacctttcaggaaaccatacccatatctgaaagtggacgacaagtgtggtgttgactccatcccggttgatcctgaaaaggttgttgcgattgtggagtccaccatgagggaccaggtcccaccaaatacgccctctgacgacatgtccagggctattgcaggtcatttggtcgagtttttcagaaacgaggtaaaacatggtaggctacctgaaaacctgctgcctttacaaagtggtataggtaacattgctaacgctgtcattgaagggcttgctggcgcccaattcaagcatttgactgtatggacggaagtgctgcaggactcgttcttggatcttttcgagaacggatctttggactacgccactgctacttccgtgagattgactgaaaagggtttcgacagagcctttgcaaactgggaaaatttcaaacacagattgtgtttgaggtctcaagtagtgtcgaacaatccggaaatgatccgtagattgggtgtcatcgccatgaataccccagtagaagttgacatttacgcgcacgccaattctacaaatgtgaatggttcccgtatgttgaacgggttgggtggatctgctgatttcttgagaaatgcaaagttgtccatcatgcatgccccctctgcaagaccaactaaagtagaccctaccggtatctctaccattgttcctatggcctctcatgtagatcaaactgagcatgacctggacatcttggtcactgaccaaggtttggcggatctaagaggtctatcgcctaaggaaagagcccgtgaaatcatcaacaagtgtgctcatcccgattatcaagctttgttgaccgattacttggacagagcagagcattacgctaaaaagcacaattgcttgcatgaaccacacatgctaaagaatgctttcaagttccacaccaacttagctgaaaagggtacaatgaaggtcgacagctgggaaccagttgactag

SEQ ID NO: 4 - protein, 526 aa, Saccharomyces boulardii
MTISNLLKQRVRYAPYLKKVKEAHELIPLFKNGQYLGWSGFTGVGTPKAVPEALIDHVEKNNLQGKLRFNLFVGASAGPEENRWAEHDMIIKRAPHQVGKPIAKAINQGRIEFFDKHLSMFPQDLTYGFYTRERKDNKILDYTIIEATAIKEDGSIVPGPSVGGSPEFITVSDKVIIEVNTATPSFEGIHDIDMPVNPPFRKPYPYLKVDDKCGVDSIPVDPEKVVAIVESTMRDQVPPNTPSDDMSRAIAGHLVEFFRNEVKHGRLPENLLPLQSGIGNIANAVIEGLAGAQFKHLTVWTEVLQDSFLDLFENGSLDYATATSVRLTEKGFDRAFANWENFKHRLCLRSQVVSNNPEMIRRLGVIAMNTPVEVDIYAHANSTNVNGSRMLNGLGGSADFLRNAKLSIMHAPSARPTKVDPTGISTIVPMASHVDQTEHDLDILVTDQGLADLRGLSPKERAREIINKCAHPDYQALLTDYLDRAEHYAKKHNCLHEPHMLKNAFKFHTNLAEKGTMKVDSWEPVD

SEQ ID NO: 5 - DNA, 1560 bp, Saccharomyces boulardii
  source 1..1560: mol_type = genomic DNA
atgttcagtagatctacgctctgcttaaagacgtctgcatcctccattgggagacttcaattgagatatttctcacaccttcctatgacagtgcctatcaagctgcccaatgggttggaatatgagcaaccaacggggttgttcatcaacaacaagtttgttccttctaaacagaacaagaccttcgaagtcattaacccttccacggaagaagaaatatgtcatatttatgaaggtagagaggacgatgtggaagaggccgtgcaggccgccgaccgtgccttctctaatgggtcttggaacggtatcgaccctattgacaggggtaaggctttgtacaggttagccgaattaattgaacaggacaaggatgtcattgcttccatcgagactttggataacggtaaagctatctcttcctcgagaggagatgttgatttagtcatcaactatttgaaatcttctgctggctttgctgataaaattgatggtagaatgattgatactggtagaacccatttttcttacactaagagacagcctttgggtgtttgtgggcagattattccttggaatttcccactgttgatgtgggcctggaagattgcccctgctttggtcaccggtaacaccgtcgtgttgaagactgccgaatccaccccattgtccgctttgtatgtgtctaaatacatcccacaggcgggtattccacctggtgtgatcaacattgtatccgggtttggtaagattgtgggtgaggccattacaaaccatccaaaaatcaaaaaggttgccttcacagggtccacggctacgggtagacacatttaccagtccgcagccgcaggcttgaaaaaagtgactttggagctgggtggtaaatcaccaaacattgtcttcgcggacgccgagttgaaaaaagccgtgcaaaacattatccttggtatctactacaattctggtgaggtctgttgtgcgggttcaagggtgtatgttgaagaatctatttacgacaaattcattgaagagttcaaagccgcttctgaatccatcaaggtgggcgacccattcgatgaatctactttccaaggtgcacaaacctctcaaatgcaactaaacaaaatcttgaaatacgttgacattggtaagaatgaaggtgctactttgattaccggtggtgaaagattaggtagcaagggttacttcattaagccaactgtctttggtgacgttaaggaagacatgagaattgtcaaagaggaaatctttggccctgttgtcactgtaaccaaattcaaatctgccgacgaagtcattaacatggcgaacgattctgaatacgggttggctgctggtattcacacctctaatattaataccgccttaaaagtggctgatagagttaatgcgggtacggtctggataaacacttataacgatttccaccacgcagttcctttcggtgggttcaatgcatctggtttgggcagggaaatgtctgttgatgctttacaaaactacttgcaagttaaagcggtccgtgccaaattggacgagtaa

SEQ ID NO: 6 - protein, 519 aa, Saccharomyces boulardii
MFSRSTLCLKTSASSIGRLQLRYFSHLPMTVPIKLPNGLEYEQPTGLFINNKFVPSKQNKTFEVINPSTEEEICHIYEGREDDVEEAVQAADRAFSNGSWNGIDPIDRGKALYRLAELIEQDKDVIASIETLDNGKAISSSRGDVDLVINYLKSSAGFADKIDGRMIDTGRTHFSYTKRQPLGVCGQIIPWNFPLLMWAWKIAPALVTGNTVVLKTAESTPLSALYVSKYIPQAGIPPGVINIVSGFGKIVGEAITNHPKIKKVAFTGSTATGRHIYQSAAAGLKKVTLELGGKSPNIVFADAELKKAVQNIILGIYYNSGEVCCAGSRVYVEESIYDKFIEEFKAASESIKVGDPFDESTFQGAQTSQMQLNKILKYVDIGKNEGATLITGGERLGSKGYFIKPTVFGDVKEDMRIVKEEIFGPVVTVTKFKSADEVINMANDSEYGLAAGIHTSNINTALKVADRVNAGTVWINTYNDFHHAVPFGGFNASGLGREMSVDALQNYLQVKAVRAKLDE

SEQ ID NO: 7 - DNA, 1503 bp, Saccharomyces boulardii
  source 1..1503: mol_type = genomic DNA
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtactttccacgacactgaatgggctacccaagacccaagagaaagaggccgtctactaagtaagttggctgatgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacggaagtcggtaagagtgttgctgtcgactcttctgaatctaacttgaagaaaatcactttggaactaggtggtaagtccgcccatttggtctttgacgatgctaacattaagaagactttaccaaatctagtaaacggtatcttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 8 - protein, 500 aa, Saccharomyces boulardii
MTKLHFDTAEPVKITLPNGLTYEQPTGLFINNKFMKAQDGKTYPVEDPSTENTVCEVSSATTEDVEYAIECADRTFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 9 - DNA, 1461 bp, Saccharomyces boulardii
  source 1..1461: mol_type = genomic DNA
atgaacgatataatcacgcaagtttctccagataatgcagagtccgctccgattctacaagaacagcaacagcaacagaactcacagtacgaaggtaacgaggaggattatggtgattcattgattcatttgaatattcaagaaaaccattattttgttacgagggaccagttgatgtctctacctgaatccctattactgtgtttatttccctcaggtgtttttttggaccgttgtggtcaggtcattgctaatttgaccagagacgatgaggtctacattgttaatttccctcctgattgttttgagtacatcatggagatatatacaaaagcgcatgatgatttgtataatcatcctgtggagaaattttttgacaggccatcaagtagctttgtttcgaatgcaaaggggttttttggactgagtagcaataattcaatttcgagcaacaatgagcaggatattttacatcaaaagcccgctattattgttttgagagaagacttggattattattgtgtacctcaggaggaatttcagtttgattctactaatgaagaaaataatgaggatttattgcgacattttatggctcaagtgaaaatggctgctggcagttatttaacttcaaaaacatcgattttccaaggtttgtattcttcgaatagactaaagcaacaacagcaacaacagaaaattgaaaaggagtccaattcttcttcaaatactaaatctacttcgaaaaaattgggacctgctgaacaacatttaatggatatgttgtgctcctccggattcactaaggaaacttgttggggtaacagaactcaagaaactggcaaaacggttataagttcactgtctctttgccgattggctaacgagacaactgaaggatttaggcaaaaatttaacgaggctaaggctaagtgggaggcagagcacaaaccttctcaagacaacttcatcaccccaatgcaatctaacatatcgattaactctttatctgcaagtaaatctaacagtaacatttctacagcaaggaatttaacaagcggaagtacagcacctgctacagcacgtgataagagaaaatcaaggctgtcgaaactagcagataacgttcgttcgcactcttcctcgagacatagttcgcagaccagaagtaaacctccggagttgcccaaattgtatgatctagtgccaaaacctaatatcaacgctaagctactattattttggagaaaacctgctcgtaaatgttggtggggtgaagaagacatagagctagaagtggaagttttcggctcttggaaagatgaatcaaagaaaatcattgaattgatcttgccaacaaacgttgatcctgaagcagaactacataaaatcattgtacccgtccgattacatattcgtagagtttggactttagagttgagcgttattggggtgcagtga

SEQ ID NO: 10 - protein, 486 aa, Saccharomyces boulardii
MNDIITQVSPDNAESAPILQEQQQQQNSQYEGNEEDYGDSLIHLNIQENHYFVTRDQLMSLPESLLLCLFPSGVFLDRCGQVIANLTRDDEVYIVNFPPDCFEYIMEIYTKAHDDLYNHPVEKFFDRPSSSFVSNAKGFFGLSSNNSISSNNEQDILHQKPAIIVLREDLDYYCVPQEEFQFDSTNEENNEDLLRHFMAQVKMAAGSYLTSKTSIFQGLYSSNRLKQQQQQQKIEKESNSSSNTKSTSKKLGPAEQHLMDMLCSSGFTKETCWGNRTQETGKTVISSLSLCRLANETTEGFRQKFNEAKAKWEAEHKPSQDNFITPMQSNISINSLSASKSNSNISTARNLTSGSTAPATARDKRKSRLSKLADNVRSHSSSRHSSQTRSKPPELPKLYDLVPKPNINAKLLLFWRKPARKCWWGEEDIELEVEVFGSWKDESKKIIELILPTNVDPEAELHKIIVPVRLHIRRVWTLELSVIGVQ

SEQ ID NO: 11 - DNA, 1923 bp, Saccharomyces cerevisiae
  source 1..1923: mol_type = genomic DNA
atgctatcgctaaaaaaatcagcgctctccaagttgactttgctcagaaacacaagaacatttacatcgtcagctttggtgcgccaaacgcagggctctgtaaacggttccgcgtccagatctgcagacgggaagtaccacataatagatcacgagtatgactgtgtggtaatcggtgccggtggtgccggccttagagcggcctttggtcttgccgaggcgggctacaagactgcttgtatatccaagcttttccccaccagatcccacactgttgctgctcagggtggtatcaatgccgctctgggaaatatgcacaaggataactggaaatggcatatgtacgatactgtgaaaggatctgattggctaggtgaccaggactccatccattacatgaccagggaagcgcccaaatcgatcattgaactggaacactatggtgttcctttttcaagaactgaaaacggtaagatctaccaaagagcctttggtggtcagaccaaggagtacggtaagggtgctcaggcctatagaacatgcgctgtcgcagacaggacaggacatgctcttttacacacgctttatggccaagctttaagacatgacacacatttctttattgagtactttgccctcgatctgttgacccataatggcgaggtcgttggtgtcatcgcttataatcaggaagacggtaccatccacagattcagagcacacaagaccattattgccactggtggctatggtagagcatacttctcttgtacctctgctcacacatgtacgggtgacggtaatgccatggtttcgcgtgctggtttccccttgcaagatttagagtttgttcaattccatccttcaggtatatatgggtctggttgcttaatcactgaaggtgctcgtggtgaaggtggttttttggttaattctgaaggtgaaagattcatggaacgttacgctcctacggccaaggatctagcttgtagagatgtcgtttccagagcaatcaccatggagatcagagaaggcagaggtgttggtaagaaaaaggaccacatgtacttacaattgagccatctacctccggaagttctaaaggaaagattgccaggtatctctgaaacagcagccatttttgctggtgtagacgtcaccaaggaacctattcccattattcctaccgtccactataacatgggtggtattcccacgaagtggaatggtgaggcattaaccattgatgaagaaactggcgaagacaaggttattcccggtttaatggcttgtggtgaagccgcttgtgtttctgtccatggtgccaatagattaggtgccaattccttgttggatcttgttgtctttggtcgtgctgttgcccatacggttgctgacactttacagcctgggttgccacacaaaccactaccttctgatttgggtaaagaatccatcgcaaacttggataaactaagaaatgctaatggttcaagatctacggcagaaattagaatgaatatgaaacaaactatgcaaaaggatgtttccgtctttagaacacaatcatctttagatgaaggtgttcggaacattactgcagtagagaagacctttgatgatgtgaagacgaccgatagatcaatgatctggaattctgacttggttgaaactctggagctacagaacttattaacctgtgcctcccaaacagctgtttccgctgctaatagaaaggaatcccgtggtgctcatgcaagagaggattatccaaatagagatgacgaacattggatgaagcatacattatcctggcaaaaggacgtcgctgccccagtgactttgaaatacagaagggttatcgatcacactttggacgaaaaggaatgtccttccgtacctccaactgtaagagcctactaa

SEQ ID NO: 12 - protein, 640 aa, Saccharomyces cerevisiae
MLSLKKSALSKLTLLRNTRTFTSSALVRQTQGSVNGSASRSADGKYHIIDHEYDCVVIGAGGAGLRAAFGLAEAGYKTACISKLFPTRSHTVAAQGGINAALGNMHKDNWKWHMYDTVKGSDWLGDQDSIHYMTREAPKSIIELEHYGVPFSRTENGKIYQRAFGGQTKEYGKGAQAYRTCAVADRTGHALLHTLYGQALRHDTHFFIEYFALDLLTHNGEVVGVIAYNQEDGTIHRFRAHKTIIATGGYGRAYFSCTSAHTCTGDGNAMVSRAGFPLQDLEFVQFHPSGIYGSGCLITEGARGEGGFLVNSEGERFMERYAPTAKDLACRDVVSRAITMEIREGRGVGKKKDHMYLQLSHLPPEVLKERLPGISETAAIFAGVDVTKEPIPIIPTVHYNMGGIPTKWNGEALTIDEETGEDKVIPGLMACGEAACVSVHGANRLGANSLLDLVVFGRAVAHTVADTLQPGLPHKPLPSDLGKESIANLDKLRNANGSRSTAEIRMNMKQTMQKDVSVFRTQSSLDEGVRNITAVEKTFDDVKTTDRSMIWNSDLVETLELQNLLTCASQTAVSAANRKESRGAHAREDYPNRDDEHWMKHTLSWQKDVAAPVTLKYRRVIDHTLDEKECPSVPPTVRAY

SEQ ID NO: 13 - DNA, 1923 bp, Saccharomyces boulardii
  source 1..1923: mol_type = genomic DNA
atgctatcgctaaaaaaatcagcgctctccaagttgactttgctcagaaacacaagaacatttacatcgtcagctttggtgcgccaaacgcagggctctgtaaacggttccgcgtccagatctgcagacgggaagtaccacataatagatcacgagtatgactgtgtggtaatcggtgccggtggtgccggccttagagcggcctttggtcttgccgaggcgggctacaagactgcttgtatatccaagcttttccccaccagatcccacactgttgctgctcagggtggtatcaatgccgctctgggaaatatgcacaaggataactggaaatggcatatgtacgatactgtgaaaggatctgattggctaggtgaccaggactccatccattacatgaccagggaagcgcccaaatcgatcattgaactggaacactatggtgttcctttttcaagaactgaaaacggtaagatctaccaaagagcctttggtggtcagaccaaggagtacggtaagggtgctcaggcctatagaacatgcgctgtcgcagacaggacaggacatgctcttttacacacgctttatggccaagctttaagatatgacacacatttctttattgagtactttgccctcgatctgttgacccataatggcgaggtcgttggtgtcatcgcttataatcaggaagacggtaccatccacagattcagagcacacaagaccattattgccactggtggctatggtagagcatacttctcttgtacctctgctcacacatgtacgggtgacggtaatgccatggtttcgcgtgctggtttccccttgcaagatttagagtttgttcaattccatccttcaggtatatatgggtctggttgcttaatcactgaaggtgctcgtggtgaaggtggttttttggttaattctgaaggtgaaagatacatggaacgttacgctcctacggccaaggatctagcttgtagagatgtcgtttccagagcaatcaccatggagatcagagaaggcagaggtgttggtaagaaaaaggaccacatgtacttacaattgagccatctacctccggaagttctaaaggaaagattgccaggtatctctgaaacagcagccatttttgctggtgtagacgtcaccaaggaacctattcccattattcctaccgtccactataacatgggtggtattcccacgaagtggaatggtgaggcattaaccattgatgaagaaactggcgaagacaaggttattcccggtttaatggcttgtggtgaagccgcttgtgtttctgtccatggtgccaatagattaggtgccaattccttgttggatcttgttgtctttggtcgtgctgttgcccatacggttgctgacactttacagcctgggttgccacacaaaccactaccttctgatttgggtaaagaatccatcgcaaacttggataaactaagaaatgctaatggttcaagatctacggcagaaattagaatgaatatgaaacaaactatgcaaaaggatgtttccgtctttagaacacaatcatctttagatgaaggtgttcggaacattactgcagtagagaagacctttgatgatgtgaagacgaccgatagatcaatgatctggaattctgacttggttgaaactctggagctacagaacttattaacctgtgcctcccaaacagctgtttccgctgctaatagaaaggaatcccgtggtgctcatgcaagagaggattatccaaatagagatgacgaacattggatgaagcatacattatcctggcaaaaggacgtcgctgccccagtgactttgaaatacagaagggttatcgatcacactttggacgaaaaggaatgtccttccgtacctccaactgtaagagcctactaa

SEQ ID NO: 14 - protein, 640 aa, Saccharomyces boulardii
MLSLKKSALSKLTLLRNTRTFTSSALVRQTQGSVNGSASRSADGKYHIIDHEYDCVVIGAGGAGLRAAFGLAEAGYKTACISKLFPTRSHTVAAQGGINAALGNMHKDNWKWHMYDTVKGSDWLGDQDSIHYMTREAPKSIIELEHYGVPFSRTENGKIYQRAFGGQTKEYGKGAQAYRTCAVADRTGHALLHTLYGQALRYDTHFFIEYFALDLLTHNGEVVGVIAYNQEDGTIHRFRAHKTIIATGGYGRAYFSCTSAHTCTGDGNAMVSRAGFPLQDLEFVQFHPSGIYGSGCLITEGARGEGGFLVNSEGERYMERYAPTAKDLACRDVVSRAITMEIREGRGVGKKKDHMYLQLSHLPPEVLKERLPGISETAAIFAGVDVTKEPIPIIPTVHYNMGGIPTKWNGEALTIDEETGEDKVIPGLMACGEAACVSVHGANRLGANSLLDLVVFGRAVAHTVADTLQPGLPHKPLPSDLGKESIANLDKLRNANGSRSTAEIRMNMKQTMQKDVSVFRTQSSLDEGVRNITAVEKTFDDVKTTDRSMIWNSDLVETLELQNLLTCASQTAVSAANRKESRGAHAREDYPNRDDEHWMKHTLSWQKDVAAPVTLKYRRVIDHTLDEKECPSVPPTVRAY

SEQ ID NO: 15 - DNA, 23 bp, synthetic construct
  source 1..23: mol_type = other DNA
ggctgattttcgcagttcggggg